(19) United States
(12) Patent Application Publication
(10) Pub. No.: US 2017/0166981 A1
(43) Pub. Date: Jun. 15, 2017

(54) BIOMARKERS AND METHODS OF USE THEREOF

(71) Applicant: THE TRANSLATIONAL GENOMICS RESEARCH INSTITUTE, Phoenix, AZ (US)

(72) Inventors: David Craig, Phoenix, AZ (US); Daniel von Hoff, Scottsdale, AZ (US); John Carpten, Phoenix, AZ (US)

(21) Appl. No.: 15/441,214

(22) Filed: Feb. 23, 2017

Related U.S. Application Data
(63) Continuation of application No. 13/983,226, filed on Oct. 10, 2013.
(60) Provisional application No. 61/438,959, filed on Feb. 2, 2011.
(60) Provisional application No. 61/444,644, filed on Feb. 18, 2011.
(60) Provisional application No. 61/472,004, filed on Apr. 5, 2011.
(60) Provisional application No. 61/490,457, filed on May 26, 2011.
(60) Provisional application No. 61/499,123, filed on Jun. 20, 2011.
(60) Provisional application No. 61/551,201, filed on Oct. 25, 2011.

(51) Int. Cl. C12Q 1/68; A61K 31/337; A61K 31/436; A61K 39/395; A61K 31/7068; A61K 31/282; C07K 16/22; A61K 31/357; A61K 31/166

(52) U.S. Cl. CPC C12Q 1/6886; A61K 31/357; A61K 31/337; A61K 31/436; A61K 31/166; A61K 31/7068; A61K 31/282; C07K 16/22; A61K 39/3955; C12Q 2600/158; C12Q 2600/156; C12Q 2600/106; C07K 2317/24

(57) ABSTRACT

Methods useful in the prediction and detection of a triple negative cancer subtype using biomarkers are provided herein.

### CROSS REFERENCES

This application is related to and claims the priority benefit of U.S. provisional application 61/438,959, filed on Feb. 2, 2011,U.S. provisional application 61/444,644, filed on Feb. 18, 2011, U.S. provisional application 61/472,004, filed on Apr. 5, 2011, U.S. provisional application 61/490,457, filed on May 26, 2011, U.S. provisional application 61/499,123, filed on Jun. 20, 2011, and U.S. provisional application 61/551,201 filed on Oct. 25, 2011, the teachings and content of which are incorporated by reference herein.

### FIELD OF INVENTION

The present invention relates to TNBC biomarker profile indicating TNBC subtype, predisposition, existence, recurrence and progression. It also provides the methods for determining sensitivity or resistance of tumor cells to particular chemotherapies.

### BACKGROUND

Analysis of genomic sequences and gene expression patterns has provided a way to improve the diagnosis and risk stratification of many diseases. For example, analysis of genomic sequences and global gene expression patterns has identified molecularly distinct subtypes of cancer in diseases that were considered homogeneous based on classical diagnostic methods. Such molecular subtypes are often associated with different clinical outcomes. Genomic aberration and global gene expression patterns can also be examined for features that correlate with clinical behavior to create prognostic signatures. Identification of genetic mutations and polynucleotides that are differentially expressed in cancer subtypes, pre-cancerous lesions, or low metastatic potential cells relative to normal cells of the same tissue type can provide the basis for diagnostic tools, facilitates drug discovery by providing for targets for candidate agents, and further serves to identify therapeutic targets for cancer therapies that are more tailored for the type or subtype of cancer to be treated.

Breast cancer is the most frequently diagnosed cancer in women and the second leading cause of cancer deaths in women. According to the World Health Organization, more than 1.2 million people will be diagnosed with breast cancer yearly worldwide, with approximately 213,000 women in the United States are diagnosed with invasive breast cancer each year (Stages I-IV). The chance of developing invasive breast cancer during a woman's lifetime is approximately 1 in 8 (about 13%). Another 62,000 women will be diagnosed with in situ breast cancer, a very early form of the disease. It is estimated that approximately 40,970 women and 460 men die from breast cancer in the United States yearly.

An important problem associated with cancer chemotherapy is that the histology of the cancer does not predict an individual's response to a given agent or a given therapeutic protocol. Global gene expression profiling has uncovered previously unrecognized subsets of human breast cancer, including the “triple-negative” subtype. Breast tumors that lack expression of estrogen receptor, progesterone receptor and Her2-neu amplification (referred to as “triple negative subtype” or “triple negative breast cancer (TNBC)”) account for approximately 15% of all breast cancer diagnoses. The TNBC subtype is among the most refractory of human breast cancers, since it cannot be treated with effective hormonal and Trastuzumab-based therapies.

Cancer management relies on a combination of initial prevention, early diagnosis, appropriate treatment, and prevention of recurrence, and genetic testing can play a role in all of these management areas. All clinical oncologists desire a strategy to use customized information to make chemotherapy recommendations that are tailored specifically to a patient's tumor characteristics. The approach has enormous intuitive appeal and is more logical to both patients and physicians than the empiric approach, whereby all patients with similar tumor type are treated according to a standardized regimen. There remains, however, a need for identification of genetic marker profiles predictive or diagnostic of particular cancer subtypes. Identification of genetic markers associated with refractory cancers may also aid in identification of key molecular pathways that may be targeted for therapeutic purposes.

### SUMMARY OF THE INVENTION

Provided herein, in part, are methods for predicting or diagnosing of a predisposition, existence, or recurrence of a triple negative cancer subtype, in particular, a triple negative subtype of breast cancer. Also provided are methods for assessing the progression of a triple negative breast cancer. Also provided herein are methods for determining sensitivity or resistance of tumor cells to particular chemotherapies, and methods for determining suitability of a therapeutic regimen for a breast cancer tumor.

Provided herein is a method for detecting existence of a triple negative breast cancer (TNBC) subtype in a tumor comprising analyzing the genome or transcriptome of a tumor tissue sample for the presence of a TNBC biomarker profile, wherein the presence of the TNBC biomarker profile indicates existence of the TNBC subtype in the tumor, wherein the TNBC biomarker profile comprises at least one alteration of the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene. In one example, the TNBC biomarker profile may comprise a deletion of exon in a RB1 gene. In some examples, the TNBC biomarker profile may comprise a deletion of exon 6 in a PTEN gene; the TNBC biomarker profile may comprise a deletion in an ERBB4 gene; the TNBC biomarker profile may comprise a mutation in an ABCB1 gene; the TNBC biomarker profile may comprise a chromosome 11 translocation and/or a chromosome 16 translocation; the TNBC biomarker profile may comprise a translocation of an SLC9A11 gene and a NCO6A gene; the TNBC biomarker profile may comprise overexpression of a gene selected from the group consisting of AURKB, FOXM1, PLK1, AURKA, BRAF, ERAS, and IQGAP3; the TNBC biomarker profile may comprise underexpression of a gene selected from the group consisting of PTEN, ERBB4, INPP4B and NOVA1; the TNBC biomarker profile may comprise copy number variation correlated with altered gene expression. In one example, the TNBC biomarker profile may comprise an overexpression of a FOXM1 gene.

Also provided is a method for treating TNBC by determining sensitivity or resistance of tumor cells to a particular chemotherapy comprising assessing a tumor tissue sample for a triple negative breast cancer (TNBC) biomarker profile, wherein the presence of TNBC subtype in the tumor sample indicates sensitivity or resistance of the tumor to a particular chemotherapy, wherein the TNBC biomarker profile comprises at least one alteration of the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene. In one example, the TNBC subtype is characterized by a biomarker profile comprising BRAF amplification and INPP4B under expression and is sensitive to treatment with combined MEK and AKT inhibitors. In another example, wherein the TNBC subtype is characterized by a biomarker profile comprising TOP2a and PBK overexpression and is sensitive to treatment with Eribulin. In yet another example, the TNBC subtype is characterized by a biomarker profile comprising IQGAP3 and AKT3 overexpression and INPP4B underexpression and is sensitive to treatment with BEZ235. In still another example, the TNBC subtype is characterized by a biomarker profile comprising ALK overexpression and is sensitive to treatment with ALK inhibitor. In one example, the TNBC subtype is characterized by a biomarker profile comprising FBXW7 and INPP4B underexpression and is sensitive to treatment with combined Taxol, Avastin and Everolimus. In a further example, wherein the TNBC subtype is characterized by a biomarker profile comprising DNA repair related gene mutations and is sensitive to treatment with combined BSI 201, Gemcitabine and Carboplatin. In another example, the TNBC subtype is characterized by a biomarker profile comprising IQGAP3 overexpression, INPP5F inversion and NEDD4 mutation and is resistant to treatment with BEZ 235. In still another example, wherein the TNBC subtype of the method is characterized by a biomarker profile comprising GART overexpression and is resistant to treatment with combined MEK and AKT inhibitors.

Further provided is a method for selecting a treatment for a subject with TNBC, and the method comprises: (a) obtaining a tumor tissue sample from the subject; (b) obtaining the TNBC biomarker profile of the sample and grouping the subject to a TNBC subtype; (c) determining sensitivity or resistance of the subject to a particular chemotherapy based on the relationship between the TNBC subtype and sensitivity or resistance to particular chemotherapies; and (d) selecting a treatment based on the TNBC subtype. The general method may further comprise administering an effective amount of both MEK and AKT inhibitors to the subject with a TNBC biomarker profile comprising BRAF amplification and INPP4B under expression. In another example, the method may further comprise administering an effective amount of Eribulin to the subject with a TNBC biomarker profile comprising IQGAP3 and AKT3 overexpression and INPP4B underexpression. The method may comprise administering an effective amount of ALK inhibitor to the subject with a TNBC biomarker profile comprising ALK overexpression. The method may also further comprise administering an effective amount of combined Taxol, Avastin and Everolimus to the subject with a TNBC biomarker profile comprising FBXW7 and INPP4B underexpression. In still another example, the general method may further comprise administering an effective amount of combined BSI 201, Gemcitabine and Carboplatin to the subject with a TNBC biomarker profile comprising DNA repair related gene mutations.

### BRIEF DESCRIPTION OF FIGURES

FIG. 1A depicts histogram illustrating mapping statistics for RNA-seq experiments for TNBC-001 and normal breast specimens. FIG. 1B depicts scatter plot illustrating correlation coefficients for RNA-seq versus microarray-based gene expression results for TNBC-001. FIG. 1C depicts correlation coefficients for specific genes measured by RNA-seq versus microarray-based gene expression differences for TNBC-001 versus normal breast samples.

FIG. 2A depicts Circos plot illustrating somatic events detected in TNBC-001, with all genes with somatic event are listed in Table 4. FIG. 2B depicts Circos plot illustrating somatic events detected TNBC-003, with all genes with somatic event are listed in Table 5. Lines adjacent to gene names describe the type of somatic event that a gene is involved in including somatic point mutation, somatic indel, copy number amplification, copy number deletion, and translocation. Lines within the Circos depict mapping of translocation events.

FIG. 3 depicts Circos plot illustrating somatic events occurring in TNBC-004, with all genes with somatic event are listed in Table 6. Lines adjacent to gene names describe the type of somatic event that a gene is involved in including somatic point mutation, somatic indel, copy number amplification, copy number deletion, and translocation. Lines within the Circos depict mapping of translocation events.

FIG. 4 depicts the allelic ratios calculated from tumor RNA libraries show increased variation compared to DNA allelic ratios suggesting the extent of functionally important regulatory variation.

FIGS. 5A-5B depict the CNV vs. RNA expression. Gene copy number (x-axis) versus gene (coding regions) coverage fold change between Tumor and Normal samples (y axis): FIG. 5A depicts DNA libraries and FIG. 5B depicts RNA libraries.

FIG. 6 depicts the MA plot highlighting the 500 most highly differentially expressed junctions resulted from differential splicing and gene fusions events.

FIG. 7 depicts the alternative splicing regulation through the overall correlation between NOVA1 depletion and its targets.

FIG. 8 depicts the whole transcriptome analysis of TNBC001.

FIG. 9 depicts the TNBC-001 PTEN Exon 6 homozygous deletion and the resulted complete protein loss in TNBC-001.

FIG. 10 depicts that NF1, PTEN and INPP4B as potential treatment targets are all in the RAS/RAF/MEK/ERK and PI3K/AKT/mTOR pathway.

FIG. 11 depicts that with lesions in both PI3K/AKT/mTOR and Ras/MEK/ERK pathways, the patient was treated with dual pathway inhibitor combination. The baseline was measured at the beginning of the treatment, after 2 cycles, and 75% regression in primary lesion was observed.

FIGS. 12A-12B depict RB1 somatic alterations validation. FIG. 12A depicts RB1 DNA mutation validation and FIG. 12B depicts RB1 RNA mutation validation.

FIG. 13 depicts integrated view of DNA and RNA fold changes within tumor for chromosome 5q31.2-31.3. For each individual, log2 fold change of RNA and DNA are shown as compared to a population-based reference sample for RNA or the germline sample or DNA. Within each, RNA is shown as red for significantly overexpressed genes (p<0.001 & foldchange >2) and DNA is shown on a blue-yellow scale where deletions are blue lines. Inaccessible regions are clear and regions not exhibiting a significant fold change are gray. Two individuals, mTNBC1 and mTNBC6, both show an approximate 150 kb homozygous deletion encompassing part of CTNNA1 leading to a significant down regulation of expression.

FIG. 14A depicts reconstruction of mTNBC2 double minute based on analysis of long-mate pair anomalous reads and copy number loss/gain calculations. Arrows show a copy number amplification is observed at breakpoints linking several segments from chromosomes 1, 7, and 12. In the lower plots, the fold change is plotted, estimated by log2(C_{G}) log2(C_{T}) where C_{G} and C_{T} are normalized coverage for germline and tumor respectively. FIG. 14B depicts BAC-FISH validation of BRAF containing double minutes in mTNBC2. BAC clones were labeled with a green fluorophor and BACs containing chromosome 7 centromeres were labeled with a red fluorophor.

### DETAILED DESCRIPTION

Somatic genomic alterations have been discovered in the triple negative subtype of breast cancer. Disclosed herein are particular genomic alterations found to be associated with TNBC and which can be used as a biomarker profile for TNBC. Such genomic alterations are useful for predictive purposes, diagnostic purposes, for methods for predicting treatment response of a tumor, methods for monitoring cancer progression, and methods for monitoring treatment progress, as described in further detail herein. Further applications of the TNBC biomarkers includes methods to determine sensitivity or resistance of tumor cells to particular chemotherapies and methods for selecting therapeutic options, as well as kits for use in the methods described herein.

### I. The Metastatic TNBC Biomarker Profile

### 1. Genomic Alterations

The genomic alterations provided herein include deletions (for example, deletions comprising exons, introns, and/or splice sites) and translocations. Within the tumor, such genomic deletions or translocations can be homozygous or heterozygous. Genomic alterations also includes transcriptome perturbations that lead to altered gene or protein expression in the tumor cells compared to normal cells, including, for example, overexpression or underexpression.

As demonstrated herein, genomic alterations identified in TNBC include, but are not limited to, an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, and/or a FBXW7 gene deletion; overexpression of AURKB, FOXM1, PLK1, AURKA genes and/or, BRAF, ERAS, and/or IQGAP3; and/or underexpression of PTEN, ERBB4, INPP4B gene and/or NOVA1 gene. Alterations can serve as biomarkers for TNBC and can be used alone, or in combination with other markers, to generate a biomarker profile for TNBC. In one embodiment, the TNBC biomarker profile includes at least one of the genomic alterations described herein. In addition, these mutations include PTEN homozygous deletion or down-regulation, INPP4B down-regulation, FBXW7 homozygous deletion, and ERAS overexpression activate PI3K/AKT/mTOR pathway, which is an event signifying specific treatment options.

2. Genomic Alteration Enriched Pathways Associated with mTNBC

(i) PI3K/AKT/m TOR Pathway

The PI3K/AKT/mTOR pathway is an intracellular signaling pathway important in apoptosis and hence cancer e.g. breast cancer and non-small-cell lung cancer. The phosphatidylinositol-3-kinase (PI3K) signaling in this pathway is crucial to many aspects of cell growth and survival. It is targeted by genomic aberrations including mutation, amplification and rearrangement more frequently than any other pathway in human cancer. The PI3K/AKT/mTOR pathway cross-talks with other pathways including the RAS, p53 and retinoblastoma (Rb) at multiple levels and constitute a signaling network implicated in tumor initiation and progression. In addition, the PI3K pathway is stimulated as a physiological consequence of many growth factors and regulators. Without the full understanding of various mechanisms, it has been found that the activation of the PI3K pathway results in a disturbance of control of cell growth and survival, which contributes to a competitive growth advantage, metastatic competence and, frequently, therapy resistance. This pathway is therefore an attractive target for the development of novel anticancer agents.

However, as it is not presently clear whether patients with aberrations of particular isoforms, alleles, or molecules in the pathway will require treatment with drugs targeting particular points in the pathway, it is important to co-develop molecular markers and targeted therapeutics. Further, an ability to pre-select patients who are likely to respond, and to identify patients not responding at an early point during treatment and triage them to alternative therapies, will greatly increase the likelihood of demonstrating efficacy and decrease the size, cost and duration of clinical trials while concurrently protecting patient life, an approach favored by drug companies and recently the FDA.

In one embodiment, the TNBC biomarker profile includes at least one of the genomic alterations activating PI3K/AKT/mTOR pathway, and these mutations may comprise PTEN homozygous deletion or down-regulation, INPP4B down-regulation, FBXW7 homozygous deletion, and ERAS overexpression.

(ii) RAS/RAF/MEK/ERK Pathway

Growth factors and mitogens use the Ras/Raf/MEK/ERK signaling cascade to transmit signals from their receptors to regulate gene expression and prevent apoptosis. Some components of these pathways are mutated or aberrantly expressed in human cancer (e.g., Ras, B-Raf). Mutations also occur at genes encoding upstream receptors (e.g., EGFR and Flt-3) and chimeric chromosomal translocations which transmit their signals through these cascades. Even in the absence of obvious genetic mutations, this pathway has been reported to be activated in over 50% of acute myelogenous leukemia and acute lymphocytic leukemia and is also frequently activated in other cancer types (e.g., breast and prostate cancers). Importantly, this increased expression is associated with a poor prognosis.

The Ras/Raf/MEK/ERK and Ras/PI3K/PTEN/AKT pathways interact with each other to regulate growth and in some cases tumorigenesis. For example, in some cells, PTEN mutation may contribute to suppression of the Raf/MEK/ERK cascade due to the ability of activated AKT to phosphorylate and inactivate different Rafs. Although both of these pathways are commonly thought to have anti-apoptotic and drug resistance effects on cells, they display different cell lineage specific effects. For example, Raf/MEK/ERK is usually associated with proliferation and drug resistance of hematopoietic cells, while activation of the Raf/MEK/ERK cascade is suppressed in some prostate cancer cell lines which have mutations at PTEN and express high levels of activated AKT.

Furthermore the Ras/Raf/MEK/ERK and Ras/PI3K/PTEN/AKT pathways also interact with the p53 pathway. For example, Raf/MEK/ERK may promote cell cycle arrest in prostate cells and this may be regulated by p53 as restoration of wild-type p53 in p53 deficient prostate cancer cells results in their enhanced sensitivity to chemotherapeutic drugs and increased expression of Raf/MEK/ERK pathway. Thus in advanced prostate cancer, it may be advantageous to induce Raf/MEK/ERK expression to promote cell cycle arrest, while in hematopoietic cancers it may be beneficial to inhibit Raf/MEK/ERK induced proliferation and drug resistance. Thus the Raf/MEK/ERK pathway has different effects on growth, prevention of apoptosis, cell cycle arrest and induction of drug resistance in cells of various lineages which may be due to the presence of functional p53 and PTEN and the expression of lineage specific factors.

The present invention provides the selection of TNBC biomarker profile utilizing genomic alteration enriched pathways such as PI3K/AKT/mTOR and Raf/MEK/ERK found herein.

In one embodiment, the TNBC biomarker profile includes a RB1 gene deletion. RB1 (Retinoblastoma-associated protein, P06400) is a key regulator of entry into cell division that acts as a tumor suppressor. In some embodiments, the RB1 gene deletion results in a loss of exon 13 from the RB1 transcript. In certain embodiments, the RB1 gene deletion occurs between exons 12 and 14 and includes a splice donor site. In certain embodiments, the RB1 gene deletion results in a loss of amino acids 406N-444Q from the RB1 polypeptide.

In one embodiment, the TNBC biomarker profile includes a PTEN gene deletion. PTEN (Mutated in multiple advanced cancers 1, P60484) is a tumor suppressor. In some embodiments, the PTEN gene deletion results in a loss of exon 6 from the PTEN transcript. In certain embodiments, the PTEN gene deletion is a deletion of approximately 1 kb encompassing exon 6. In some embodiments, the PTEN deletion is homozygous in the TNBC. In some embodiments, the PTEN gene deletion results in a frameshift mutation and premature truncation of the PTEN polypeptide. In certain embodiments, the PTEN gene deletion results in a loss of exon 6 bases 493-634 from the PTEN transcript.

In one embodiment, the TNBC biomarker profile includes an ERBB4 gene deletion. ERBB4 (Receptor tyrosine-protein kinase erbB-4, Q15303) plays an essential role as cell surface receptor for neuregulins and EGF family members. In some embodiments, the ERBB4 gene deletion includes the deletion of approximately 4.3 kb of the ERBB4 gene and is intronic.

In one embodiment, the TNBC biomarker profile includes an ABCB1 gene mutation. ABCB1 (Multidrug resistance protein 1, P08183) are reported to be associated with susceptibility to inflammatory bowel disease

In one embodiment, the TNBC biomarker profile includes a chromosome 11 translocation and/or a chromosome 16 translocation.

In one embodiment, the TNBC biomarker profile includes an SLC9A11 gene translocation and/or a NCOA6 gene translocation. NCOA6 (Nuclear receptor coactivator 6, Q14686) directly binds nuclear receptors and stimulates the transcriptional activities in a hormone-dependent fashion. In some embodiments, the TNBC biomarker profile includes an SLC9A11-NCOA6 translocation. In certain embodiments, the SLC9A11-NCOA6 translocation results in a fusion transcript from the two genes. In some embodiments, the SLC9A11-NCOA6 translocation results in a fusion transcript comprising exons 2-9 of NCOA6 and exons 22-27 of SLC9A11. SLC9A11 (Sodium/hydrogen exchanger 11, Q5TAH2). SLC9A11 is reported to be involved in pH regulation.

In one embodiment, the TNBC biomarker profile includes a NF1 gene deletion. NF1 (Neurofibromin, P21359) Stimulates the GTPase activity of Ras. Defects in NF1 are the cause of many diseases including neurofibromatosis type 1 (NF1) and colorectal cancer. In some embodiment, the deletion is homozygous. In some embodiment, the NF1 gene deletion includes the homozygous deletion of a 19 bp coding region.

In one embodiment, the TNBC biomarker profile includes a FBXW7 gene deletion. FBXW7 (F-box/WD repeat-containing protein 7, Q969H0) is reported to recognize and bind to phosphorylated target proteins. In some embodiment, the FBXW7 gene deletion includes a homozygous deletion.

In one embodiment, the TNBC biomarker profile includes over expression of AURKB, FOXM1, PLK1, AURKA, BRAF, ERAS and/or IQGAP3 genes in the tumor tissue relative to normal cells of the same type of tissue. In another embodiment, the TNBC biomarker profile includes underexpression of PTEN, ERBB4, INPP4B and/or NOVA1 genes in the tumor tissue relative to normal cells of the same type of tissue.

In certain embodiments, the TNBC biomarker profile includes an over expression of a AURKB gene. AURKB is a serine/threonine-protein kinase component of the chromosomal passenger complex (CPC), a complex that acts as a key regulator of mitosis. The CPC complex has essential functions at the centromere in ensuring correct chromosome alignment and segregation and is required for chromatin-induced microtubule stabilization and spindle assembly. It is also involved in the bipolar attachment of spindle microtubules to kinetochores and is a key regulator for the onset of cytokinesis during mitosis.

In certain embodiments, the TNBC biomarker profile includes an over expression of a FOXM1 gene. FOXM1 is a forkhead transcription factor whose loss has been reported to lead to centrosome amplification and mitotic catastrophe in certain cancer cells, such as primary breast cancer (Wonsey et al., Cancer Research, 65:5181 (2005)). FOXM1 is a key regulator of cell proliferation and is overexpressed in a variety of primary cancer forms, such as breast cancer, cell carcinoma and hepatocellular carcinoma (Kwok, Molecular Cancer Research, 8:24 (2010)). The over expression of FOXM1 has been reported to mediate breast cancer resistance to treatment with Herceptin and Paclitaxel (Carr, Cancer Research, 70:5054 (2010)).

In certain embodiments, the TNBC biomarker profile includes an over expression of a PLK1 gene. PLK1 performs several important functions throughout M phase of the cell cycle, including the regulation of centrosome maturation and spindle assembly, the removal of cohesins from chromosome arms, the inactivation of anaphase-promoting complex/cyclosome (APC/C) inhibitors, and the regulation of mitotic exit and cytokinesis.

In certain embodiments, the TNBC biomarker profile includes an over expression of a AURKA gene. AURKA is a mitotic serine/threonine kinases that contributes to the regulation of cell cycle progression.

In certain embodiments, the TNBC biomarker profile includes an over expression of a BRAF gene. BRAF is involved in the transduction of mitogenic signals from the cell membrane to the nucleus. In certain embodiments, the TNBC biomarker profile includes an over expression of a ERAS gene. ERAS is a Ras protein that binds GDP/GTP and possess intrinsic GTPase activity. ERAS plays an important role in the tumor-like growth properties of embryonic stem cells.

In certain embodiments, the TNBC biomarker profile includes an over expression of a ERAS gene. ERAS is a Ras GTPase-activating-like protein, and is an oncogenic kinase.

In certain embodiments, the TNBC biomarker profile includes an under expression of a PTEN gene. PTEN is a tumor suppressor. It acts as a dual-specificity protein phosphatase, dephosphorylating tyrosine-, serine- and threonine-phosphorylated proteins. It also acts as a lipid phosphatase, removing the phosphate in the D3 position of the inositol ring from phosphatidylino sitol 3,4,5-trisphosphate, phosphatidylinositol 3,4-diphosphate, phosphatidylinositol 3-phosphate and inositol 1,3,4,5-tetrakisphosphate.

In certain embodiments, the TNBC biomarker profile includes an under expression of a ERBB4 gene. ERBB4 is a tyrosine-protein kinase that plays an essential role as cell surface receptor for neuregulins and EGF family members and regulates development of the heart, the central nervous system and the mammary gland, gene transcription, cell proliferation, differentiation, migration and apoptosis.

In certain embodiments, the TNBC biomarker profile includes an under expression of a INPP4B gene. INPP4B catalyzes the hydrolysis of the 4-position phosphate of phosphatidylinositol 3,4-bisphosphate, inositol 1,3,4-trisphosphate and inositol 1,4-bisphosphate.

In certain embodiments, the TNBC biomarker profile includes an under expression of a NOVA1 gene. NOVA1 may regulate RNA splicing or metabolism in a specific subset of developing neurons.

In one embodiment, the TNBC biomarker profile includes any one or a combination of two or more of the genomic alterations described herein.

In one embodiment, the TNBC biomarker profile includes at least two of the following genomic alterations: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; underexpression of PTEN, ERBB4, INPP4B gene and/or NOVA1 gene.

In one embodiment, the TNBC biomarker profile includes at least three of the following genomic alterations: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; underexpression of PTEN, ERBB4, INPP4B gene and/or NOVA1 gene.

In one embodiment, the TNBC biomarker profile includes at least four of the following genomic alterations: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; underexpression of PTEN, ERBB4, INPP4B gene and/or NOVA1 gene.

In one embodiment, the TNBC biomarker profile includes at least two of: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion. In another embodiment, the TNBC biomarker profile includes at least two of the following genomic alterations: overexpression of AURKB, FOXM1, PLK1, AURKA genes, BRAF, ERAS, IQGAP3; and/or underexpression of PTEN, ERBB4, INPP4B gene and/or NOVA1 gene.

### II. The Use of the TNBC Biomarker Profile Thereof

In one aspect, provided herein are methods for predicting a predisposition, existence, or recurrence of a triple negative cancer phenotype, for example, a triple negative subtype of breast cancer. Such methods comprise assessing a sample of the suspect cancer tissue for the triple negative biomarker profile as described herein. With regard to a breast cancer tumor or suspect tissue sample, determination of the presence of the TNBC biomarker profile indicates a predisposition of the tumor to become a TNBC subtype or determination of the presence of the TNBC biomarker profile indicates that a TNBC subtype exists or has recurred in the tumor or suspect tissue

In one embodiment, a method is provided for predicting a predisposition of a tumor to be a TNBC subtype comprising analyzing the genome or transcriptome of a tumor tissue sample for the presence of a TNBC biomarker profile as described herein, wherein the presence of the TNBC biomarker profile indicates a predisposition of the tumor to be a TNBC subtype.

In one embodiment, a method is provided for predicting the existence of a TNBC subtype in a tumor comprising analyzing the genome or transcriptome of the tumor tissue sample for the presence of a TNBC biomarker profile as described herein, wherein the presence of the TNBC biomarker profile indicates existence of the TNBC subtype in the tumor.

In one embodiment, provided is a method for predicting of a predisposition of a tumor to be a TNBC subtype comprising analyzing the genome, transcriptome, or polypeptides of a tumor tissue sample for the presence of a TNBC biomarker profile comprising at least two of the following alterations: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B gene and/or NOVA1 gene.

In one embodiment, provided is a method for predicting existence of a TNBC subtype in a tumor comprising analyzing the genome, transcriptome or polypeptides of a tumor tissue sample for the presence of a TNBC biomarker profile comprising at least two of the following alterations: an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; underexpression of PTEN, ERBB4, INPP4B gene and/or NOVA1 gene, wherein the presence of the TNBC biomarker profile indicates existence of the TNBC subtype in the tumor.

In another aspect, provided herein are methods for assessing the progression of a TNBC in a subject by detecting the level of a TNBC biomarker profile in a first sample from the subject at a first period of time, detecting the level of a TNBC biomarker profile in a second sample from the subject at a second period of time and comparing the level of the TNBC biomarker profiles. In some embodiments, the first sample is taken from the subject prior to being treated for the TNBC and the second sample is taken from the subject after being treated for the cancer.

### 1. The TNBC Biomarker and Gene Alleles

Genomic alterations provided herein including deletions (for example, deletions comprising exons, introns, and/or splice sites) and translocations, whether homozygous or heterozygous. Genomic alterations also includes transcriptome perturbations that lead to altered gene or protein expression in the tumor cells compared to normal cells, including, for example, overexpression or underexpression. Methods, reagents, equipment, and software for genome and transcriptome sequencing and analysis are known in the art and commercially available. Methods, reagents, and equipment for detecting and/or measuring the amount of the TNBC biomarker nucleic acids and proteins are known in the art and commercially available.

Different expression may be because of various gene alleles in metastatic triple negative breast cancer (mTNBC) and non-metastatic TNBC cell. An allele includes any form of a particular nucleic acid that may be recognized as a form of the particular nucleic acid on account of its location, sequence, expression level, expression specificity or any other characteristic that may identify it as being a form of the particular gene. Alleles include but need not be limited to forms of a gene that include point mutations, silent mutations, deletions, frameshift mutations, single nucleotide polymorphisms (SNPs), inversions, translocations, heterochromatic insertions, and differentially methylated sequences relative to a reference gene, whether alone or in combination. An allele of a gene may or may not produce a functional protein; may produce a protein with altered function, localization, stability, dimerization, or protein-protein interaction; may have overexpression, underexpression or no expression; may have altered temporal or spacial expression specificity.

An allele may be compared to another allele that may be termed a wild type form of an allele. In comparison to the wild type allele, a different allele may be called a mutation or a mutant. Mutants may also be interchangeably called variants. In some cases, the wild type allele is more common than the mutant. A genetic mutation or variance may be any detectable change in genetic material such as DNA, or a corresponding change in the RNA or protein product of that genetic material. In the example of gene mutation, the DNA sequence of a gene or any controlling elements surrounding the gene is altered. Controlling elements include promoter, enhancer, suppressor or silencing elements capable of controlling a given gene. Other examples of mutations include alterations in the products of gene expression such as RNA or protein that result from corresponding mutations in the DNA.

Conserved variants encompass any mutation or other variant in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Depending on the location of the variance in the overall context of the protein, some substitution may have little or no effect on the apparent molecular weight or isoelectric point of the protein or polypeptide.

Amino acids other than those indicated as conserved may differ in a protein or enzyme so that the percent protein or amino acid sequence similarity between any two proteins of similar function may vary and may be, for example, from about 70% to about 99% as determined according to an alignment scheme such as by the Cluster Method, wherein similarity is based on the MEGALIGN algorithm. The concept of a variant further encompasses a polypeptide or enzyme which has at least 60%, 75%, 85%, 90%, or 95% amino acid identity as determined by algorithms such as BLAST or FASTA and which has the same or substantially similar properties and/or activities as the native or parent protein or enzyme to which it is compared.

Another example of gene variant is a gain-of-function variant. Gain-of-function variants of polypeptides encompass any variant in which a change in one or more amino acid residues in a protein or enzyme improves the activity of the polypeptide. Examples of activities of a polypeptide that may be improved by a change resulting in a gain of function variant include but are not limited to enzymatic activity, binding affinity, phosphorylation or dephosphorylation efficiency, activation, deactivation, or any other activity or property of a protein that may be quantitatively measured by some method now known or yet to be disclosed.

The presence or absence of an allele may be detected through the use of any process known in the art, including using primers and probes designed according to a specific allele for PCR, sequencing, hybridization analyses.

### 2. Biomarker Expression Detection

One aspect of this invention provides a method of identifying subjects for treatment with specific chemo agents, and excluding subjects with chemo-resistance from chemotherapy using biomarkers. Specifically, the biomarkers disclosed in this invention include overexpression of AURKB, FOXM1, PLK1, AURKA genes, BRAF, ERAS, IQGAP3; underexpression of PTEN, ERBB4, INPP4B gene and/or NOVA1 gene, all of which are associated to mTNBC.

The expression of a biomarker in a sample may be more or less than that of a predetermined level to predict the presence or absence of a cellular or physiological characteristic. The expression of the biomarker or target in the test subject may be 1,000,000×, 100,000×, 10,000×, 1000×, 100×, 10×, 5×, 2×, 1×, 0.5×, 0.1×, 0.01×, 0.001×, 0.0001×, 0.00001×, 0.000001×, or 0.0000001× of the predetermined level indicting the presence or absence of a cellular or physiological characteristic. The predetermined level of expression may be derived from a single control sample or a set of control samples.

The expression level of a biomarker can be determined, for example, by comparing mRNA or protein level in a test subject or sample to a control. In one embodiment of this invention, the comparison is between the test subject or sample and the paired subject or sample that is without cancerous cells. In one embodiment, the expression of the biomarker in a sample may be compared to a control level of expression predetermined to predict the presence or absence of a particular physiological characteristic. The predetermined control level of biomarker expression may be derived from a single control or a set of controls. Alternatively, a control may be a sample having a previously determined control level of expression of a specific biomarker. Comparison of the expression of the biomarker in the sample to a control level of expression results in a prediction that the sample exhibits or does not exhibit the cellular or physiological characteristic.

Expression of a biomarker may be assessed by any number of methods used to detect material derived from a nucleic acid template used currently in the art and yet to be developed. Examples of such methods include any biomarker nucleic acid detection method such as the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, reverse transcriptase treatment followed by direct sequencing. Other examples include any method of assessing biomarker protein expression such as flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay.

Other methods used to assess biomarker expression include the use of natural or artificial ligands capable of specifically binding a biomarker or a target. Such ligands include antibodies, antibody complexes, conjugates, natural ligands, small molecules, nanoparticles, or any other molecular entity capable of specific binding to a target. The term “antibody” is used herein in the broadest sense and refers generally to a molecule that contains at least one antigen binding site that immunospecifically binds to a particular antigen target of interest. Antibody thus includes but is not limited to native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term thus includes full length antibodies and/or their variants as well as immunologically active fragments thereof, thus encompassing, antibody fragments capable of binding to a biological molecule (such as an antigen or receptor) or portions thereof, including but not limited to Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

Ligands may be associated with a label such as a radioactive isotope or chelate thereof, dye (fluorescent or nonfluorescent,) stain, enzyme, metal, or any other substance capable of aiding a machine or a human eye from differentiating a cell expressing a target from a cell not expressing a target. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

In addition, biomarker differential expression encompasses any detectable difference between the expression of a biomarker in one sample relative to the expression of the biomarker in another sample. Differential expression may be assessed by a detector, an instrument containing a detector, or by aided or unaided human eye. Examples include but are not limited to differential staining of cells in an IHC assay configured to detect a target, differential detection of bound RNA on a microarray to which a sequence capable of binding to the target is bound, differential results in measuring RT-PCR measured in ACt or alternatively in the number of PCR cycles necessary to reach a particular optical density at a wavelength at which a double stranded DNA binding dye (e.g. SYBR Green) incorporates, differential results in measuring label from a reporter probe used in a real-time RT-PCR reaction, differential detection of fluorescence on cells using a flow cytometer, differential intensities of bands in a Northern blot, differential intensities of bands in an RNAse protection assay, differential cell death measured by apoptotic markers, differential cell death measured by shrinkage of a tumor, or any method that allows a detection of a difference in signal between one sample or set of samples and another sample or set of samples.

Techniques using microarrays may also be advantageously implemented to detect genetic abnormalities or assess gene expression. Gene expression may be that of the one or more biomarkers chosen from AURKB, FOXM1, PLK1, AURKA, BRAF, ERAS, IQGAP3, PTEN, ERBB4, INPP4B, NOVA1 gene or the expression of another set of genes upstream or downstream in a pathway of which the one or more biomarkers is a component or a regulator. In one embodiment, microarrays may be designed so that the same set of identical oligonucleotides is attached to at least two selected discrete regions of the array, so that one can easily compare a normal sample, contacted with one of said selected regions of the array, against a test sample, contacted with another of said selected regions. Examples of microarray techniques include those developed by Nanogen, Inc. (San Diego, Calif.) and those developed by Affymetrix (Santa Clara, Calif.). However, all types of microarrays, also called “gene chips” or “DNA chips”, may be adapted for the identification of mutations. Such microarrays are well known in the art.

In one embodiment of detecting the presence of a biomarker associated with a characteristic of a disease, a threshold value may be obtained by performing the one or more above mentioned assays on samples obtained from a population of patients having a certain disease condition (chemo-resistant cancer, for example) and from a second population of subjects that do not have the disease condition. In assessing disease outcome or the effect of treatment, a population of patients with a disease condition may be followed for a period of time. After the period of time expires, the population may be divided into two or more groups based on one or more parameters. For example, the population may be divided into a first group of patients whose disease progresses to a particular endpoint and a second group of patients whose disease does not progress to the particular endpoint. Examples of endpoints include disease recurrence, death, metastasis, chemo response or resistance, or other clinically meaningful indexes. Based on the observation of the parameters, a predetermined level of expression of a biomarker for each group may be selected to signify a particular physiological or cellular characteristic including identifying or diagnosing a particular disease, assessing a risk of outcome or a prognostic risk, or assessing the risk that a particular treatment will or will not be effective. If expression of the biomarker in a test sample is more similar to the predetermined expression of the biomarker in one group relative to the other group, the sample may be assigned a risk of having the same outcome as the patient group to which it is more similar.

Additionally, a predetermined level of biomarker expression may be established by assessing the expression of a biomarker in a sample obtained first from one patient, assessing the expression of the biomarker in additional samples obtained later in time from the same patient, and comparing the expression of the biomarker from the samples later in time with the previous sample(s). This method may be used in the case of biomarker that indicates, for example, progression or worsening of disease or lack of efficacy of a treatment regimen or remission of a disease or efficacy of a treatment regimen.

In a preferred embodiment, predicting a test sample or subject's response to a therapy, such as a drug therapy, is based on the detection of an altered expression of a biomarker in the test subject in comparison to the expression level in a subject responsive to the therapy, and the one or more biomarkers is selected from AURKB, FOXM1, PLK1, AURKA, BRAF, ERAS, IQGAP3, PTEN, ERBB4, INPP4B, NOVA1.

3. TNBC Biomarker Profile Guided mTNBC Therapy

Identification of a tumor as a TNBC subtype would provide valuable information for determining treatment regimens and options for the patient. In another aspect, provided herein are methods for determining sensitivity or resistance of tumor cells to particular chemotherapies and methods for determining suitability of a therapeutic regimen for a breast cancer tumor. Such methods comprise assessing a tumor tissue sample for the TNBC biomarker profile described herein, wherein the presence of TNBC subtype in the tumor sample would indicate the sensitivity or resistance of the tumor to particular chemotherapies and/or that particular therapeutic regimens with little or no therapeutic value to the patient. Such a determination would save the patient valuable time and resources.

Studies have indicated that the triple negative subtype of breast cancer may disproportionally affect African American women and that women of black African ancestry may comprise a high-risk population for TNBC. Accordingly, in certain embodiments, the methods provided relate to subjects that include black African ancestry such as populations comprising persons of African descent or lineage. Black African ancestry may be determined by self reporting as African-Americans, Afro-Americans, Black Americans, or being a member of the black race. For example, African Americans or Black Americans are those persons living in North America and having origins in any of the black racial groups of Africa. In another example, self-reported persons of black African ancestry may have at least one parent of black African ancestry or at least one grandparent of black African ancestry.

Examples of chemotherapy medications for breast cancer include: Abraxane (chemical name: paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), Xeloda (chemical name: capecitabine). In many cases, chemotherapy medicines are given in combination, known as chemotherapy regimens. In early stage breast cancer, standard chemotherapy regimens lower the risk of the cancer coming back. In advanced breast cancer, chemotherapy regimens make the cancer shrink or disappear in about 30-60% of people treated. However, every cancer responds differently to chemotherapy, and many are resistant to one or more chemotherapy agents.

In one embodiment, the presence of TNBC subtype in the tumor sample characterized by a biomarker profile comprising BRAF amplification and INPP4B under expression indicates sensitivity to treatment with combined MEK and AKT inhibitors. In one embodiment, the MEK inhibitor is GSK1120212, and the AKT inhibitor is GSK2141795.

In one embodiment, the presence of TNBC subtype in the tumor sample characterized by a biomarker profile comprising TOP2a and PBK overexpression indicates sensitivity to treatment with Eribulin. TOP2a (DNA topoisomerase 2-alpha, P11388) controls topological states of DNA by transient breakage and subsequent rejoining of DNA strands. Topoisomerase II makes double-strand breaks. PBK (Lymphokine-activated killer T-cell-originated protein kinase, Q96KB5), when phosphorylated, forms a complex with TP53, leading to TP53 destabilization and attenuation of G2/M checkpoint during doxorubicin-induced DNA damage.

In one embodiment, the presence of TNBC subtype in the tumor sample characterized by a biomarker profile comprising IQGAP3 and AKT3 overexpression and INPP4B underexpression indicates sensitivity to treatment with BEZ235. AKT3 (RAC-gamma serine/threonine-protein kinase, Q9Y243) regulates many processes including metabolism, proliferation, cell survival, growth and angiogenesis through serine and/or threonine phosphorylation of a range of downstream substrates.

In one embodiment, the presence of TNBC subtype in the tumor sample characterized by a biomarker profile comprising ALK overexpression indicates sensitivity to treatment with ALK inhibitor including LDK378. ALK (ALK tyrosine kinase receptor, Q9UM73) plays an important role in the genesis and differentiation of the nervous system.

In one embodiment, the presence of TNBC subtype in the tumor sample characterized by a biomarker profile comprising FBXW7 and INPP4B underexpression indicates sensitivity to treatment with combined Taxol, Avastin and Everolimus.

In one embodiment, the presence of TNBC subtype in the tumor sample characterized by a biomarker profile comprising DNA repair related gene mutations indicates sensitivity to treatment with combined BSI 201, Gemcitabine and Carboplatin.

In one embodiment, the presence of TNBC subtype in the tumor sample characterized by a biomarker profile comprising IQGAP3 overexpression, INPP5F inversion and NEDD4 mutation indicates resistance to treatment with BEZ 235.

In one embodiment, the presence of TNBC subtype in the tumor sample characterized by a biomarker profile comprising GART overexpression indicates resistance to treatment with combined MEK and AKT inhibitors.

Examples of current investigational or off-label cancer drugs under the TNBC biomarker guided treatment include, but not limited to, BEZ235, or NVP-BEZ235, an PI3K inhibitor being investigated as a possible cancer treatment; velcade, a drug approved for the treatment of multiple myeloma and mantle cell lymphoma (a cancer of lymph nodes); pemitrexed, approved to be used alone or with other drugs to treat malignant pleural mesothelioma in patients who cannot be treated with surgery and advanced or metastasized non-small cell lung cancer; ENZ2208, for advanced cancers; Curcumin, being investigated for treating multiple myeloma, pancreatic cancer, myelodysplastic syndromes, colon cancer, psoriasis, and Alzheimer's disease; Everolimus, currently used as an immunosuppressant to prevent rejection of organ transplants and treatment of renal cell cancer; BSI 201, an investigational anticancer agent being studied in multiple cancers, including Phase II and Phase III clinical trials in breast, lung and ovarian cancers.

### III. Kits

The invention further provides kits that facilitate the detection of altered expression of one or more biomarkers associated to mTNBC. The kit may comprise one or more reagents to identify a patient for elimination from a chemotherapy treatment. The reagents in the kit may be primers, probes, and/or antibodies that are capable of identifying a biomarker or target associated to mTNBC.

The kit that facilitates nucleic acid based assays may further comprise one or more of the following: nucleic acid extraction reagents, controls, disposable cartridges, labeling reagents, enzymes including PCR amplification reagents such as the DNA polymerases Taq or Pfu, reverse transcriptase, or one or more other polymerases, and/or reagents that facilitate hybridization.

In another embodiment, the kit may further comprise a label that can be used to label the primer or probe oligonucleotide. A label may be any substance capable of aiding a machine, detector, sensor, device, or enhanced or unenhanced human eye from differentiating a sample that that displays positive expression from a sample that displays reduced expression. Examples of labels include but are not limited to: a radioactive isotope or chelate thereof, a dye (fluorescent or nonfluorescent,) stain, enzyme, or nonradioactive metal. Specific examples include but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylaminophenylazo) benzoic acid (“Dabcyl”); 4-(4′-dimethylamino-phenylazo)sulfonic acid (sulfonyl chloride) (“Dabsyl”); 5-((2-aminoethyl)-amino)-naphtalene- 1-sulfonic acid (“EDANS”); Psoralene derivatives, haptens, cyanines, acridines, fluorescent rhodol derivatives, cholesterol derivatives; ethylene diamine tetra-acetic acid (“EDTA”) and derivatives thereof or any other compound that signals the presence of the labeled nucleic acid. In one embodiment of the invention, the label includes one or more dyes optimized for use in genotyping. Examples of such dyes include but are not limited to: dR110, 5-FAM, 6FAM, dR6G, JOE, HEX, VIC, TET, dTAMRA, TAMRA, NED, dROX, PET, BHQ+, Gold540, and LIZ.

In yet anther embodiment, the primers and probes in the kit may have been labeled, and can be applied without labeling process in PCR, sequencing reaction, or binding to a solid substrate such as oligonucleotide array.

The kit that facilitates the detection of altered expression of one or more biomarkers or targets associated to mTNBC may also comprise instructions for use. In one embodiment, the kit may further comprise an indication that links the output of the assays provided by the kit to a particular result. For example, an indication may provide guide to associate the presence or absence of one or more sequences to a specific treatment plan. The output of the assay may be in a form of a particular sequence, a particular genotype, a particular expression level in a real-time quantitative PCR reaction, a level of fluorescence or radioactive decay, a value derived from a standard curve, or from a positive or negative control, or any combination of these and other outputs. The indication may be printed on a writing that may be included in the kit or it may be posted on the Internet or embedded in a software package. The writing may include graphical depictions of results such as a photomicrograph or amplification plot.

The kit that facilitate the detection of altered expression of one or more biomarkers or targets associated to mTNBC may further comprise a device used to collect the sample. Such devices may include but need not be limited to: swabs, needles, blood collection tubes, wipes, or any other apparatus that may be used to collect a biological sample from a subject.

### EXAMPLE

The following examples are provided by way of illustration and not by way of limitation.

### Example 1

### The Whole-Genome and Transcriptome Sequencing Results of One Patient Revealed Genomic alterations in the TNBC

Samples of TNBC tumor and non-tumor normal tissue were obtained from an African American female subject (age 53) for whole-genome and transcriptome sequencing and sequence analysis. The subject's primary treatment was adjuvant therapy (anthracyclines and taxanes followed by paclitaxel (AC/T). In response, the tumor “melted away” with cycle 1 of AC and at definitive surgery, the subject had a pathologic complete response with no cancer in her breast or axillary lymph nodes. Two years later the subject presented with a very rapidly growing ipsilateral internal mammary lympho node protruding from her chest and invading into her sternum as was as some other mediastinal lymph nodes. The initial disease responded to a secondary treatment of gemcitabine/carboplatin plus iniparib (PARP inhibitor) for 8 cycles (6 months). The subject has not had BRCA½ testing.

Tissue sample preparation, sequencing, and analysis was performed using the SOLiD™ 4.0 System (Applied Biosystems) and manufacture's protocols.

For the whole-genome sequencing study, four simultaneous SOLiD™ 4 System runs were performed with 50 bp×50 bp mate-pairs, 1.5 kb inserts, and two slides per run. A total of eight slides were run. The tumor and germline genomes were sequenced and analyzed using analysis tools (including BFAST and SOLiD™ BioScope™ Software (Applied Biosystems)) to uncover somatic alterations.

For the transcriptome sequencing, libraries were prepared from RNA of the tissue samples and two libraries per sample were processed using one 50 bp fragment library per quad and 50 bp×35 bp paired end runs later. Libraries were prepared from two ethnicity-matched population controls of hyperplastic breast tissue and two replicates per sample were processed. Tumor and control transcriptomes (RNA-seq) were sequenced and analyzed using tools including SOLiD™ BioScope™ Software, EdgeR, and DESeq to compare RNA expression.

Analysis of the whole-genome and transcriptome sequencing results revealed genomic alterations in the TNBC tumor tissue compared with the normal tissue, including, for example, point mutations, small indels, copy number alterations (e.g. gains and/or losses), and translocations (see Table 1). Some somatic alterations associated with the TNBC included exon deletions, intronic deletions and translocations. The transcriptome analysis revealed consequences of a number of the genomic alterations including exon skipping and the generation of an apparent fusion transcript. In addition, transcriptome analysis revealed differential gene expression between the TNBC and normal tissue, including transcriptome perturbations in cell cycle gene expression.

TABLE 1
Genomic alterations in a TNBC patient using the whole-genome and
transcriptome sequencing

| | | UniProtKB/ | | |
|---|---|---|---|---|
| | | Swiss- | | |
| Gene | Protein | Prot ID | Somatic | |
| Name | Name | Number | alteration | Phenotype |
| RB1 | Retinoblastoma- | P06400 | deletion in the | results in the |
| | associated | | RB1 gene | loss of exon 13 |
| | protein 1 | | occurs | from the RB1 |
| | | | between | transcript. |
| | | | exons 12 and | Exon 12 is |
| | | | 14 and | joined in |
| | | | includes a | frame with |
| | | | splice donor | exon 14. |
| | | | site | |
| PTEN | Phosphatase | P60484 | a homozygous | results in |
| | and tensin | | deletion of | deletion of |
| | homolog | | approximately | exon 6 bases |
| | | | 1 kb | 493-634 from |
| | | | encompassing | the transcript |
| | | | exon 6 | and in a |
| | | | | frameshift |
| | | | | mutation and |
| | | | | premature |
| | | | | truncation |
| | | | | of the |
| | | | | polypeptide. |
| ERBB4 | Receptor | Q15303 | An intronic | results in a loss |
| | tyrosine-protein | | deletion of | of expression |
| | kinase erbB-4 | | approximately | of the |
| | | | 4.3 kb | ERBB4 gene |
| ABCB1 | Multidrug | P08183 | a single | |
| | resistance | | nucleotide | |
| | protein 1 | | mutation | |
| NCOA6 | Nuclear | Q14686 | Translocation | a fusion |
| | receptor | | and fusion | transcript |
| | coactivator 6 | | | comprising |
| | | | | exons 2-9 of |
| | | | | NCOA6 and |
| | | | | Exons 22-27 |
| | | | | of SLC9A11. |
| SLC9A11 | Sodium/ | Q5TAH2 | Translocation | a fusion |
| | hydrogen | | and fusion | transcript |
| | exchanger | | | comprising |
| | 11 | | | exons 2-9 of |
| | | | | NCOA6 and |
| | | | | Exons 22-27 |
| | | | | of SLC9A11. |

One genomic alteration identified in the TNBC is a deletion in the Retinoblastoma-associated protein 1 (RB 1) gene. Specifically, the deletion in the RB1 gene occurs between exons 12 and 14 and includes a splice donor site. This deletion results in exon skipping and the loss of exon 13 from the RB1 transcript. With the deletion at RB1 1288-1332+2de147, exon 12 is joined in frame with exon 14 and amino acids 406N-444Q are deleted from the RB1 polypeptide.

Another genomic alteration identified in the TNBC is a homozygous deletion of approximately 1 kb encompassing exon 6 of the phosphatase and tensin homolog (PTEN) gene. The PTEN gene deletion results deletion of exon 6 bases 493-634 from the transcript and in a frameshift mutation and premature truncation of the polypeptide. The frameshift mutation occurs after G165 and the polypeptide truncates at amino acid 173. PTEN was significantly underexpressed in the TNBC tissue. Immunohistochemical analysis of the TNBC tissue also demonstrates that the truncating mutation results in loss of the PTEN protein.

Another genomic alteration identified in the TNBC is a deletion of approximately 4.3 kb in the ERBB4 gene. The ERBB4 deletion is intronic and results in a loss of expression of the ERBB4 gene.

Another genomic alteration identified in the TNBC is a single nucleotide mutation of the ABCB1 gene.

Another genomic alteration identified in the TNBC are translocations including chromosome 11 and chromosome 16.

Another genomic alteration identified in the TNBC are apparent translocations with somatic discordant mate-pairs mapping to chromosome 1 (SLC9A11) and chromosome 20 (NCOA6). The RNA-seq data from the transcriptome analysis provides strong evidence for a fusion transcript comprising exons 2-9 of NCOA6 and Exons 22-27 of SLC9A11.

In addition, differential gene expression between the TNBC and normal tissue was found for a number of genes, including up-regulation and down-regulation of gene expression. For example, AURKB, FOXM1, PLK1 and AURKA were found to be over-expressed in the tumor tissue (see further in Example 2).

From the patient described here in Example 1, 1.5 kb mate pair libraries from tumor and normal tumor tissue were sequenced on SOLiD™ version 4 (Applied Biosystems) to a depth of 30× mapped using 50mer reads. Tumor tissue purity was estimated at 95% tumor content. The tumor transcriptome was sequenced to 120 million reads comprising four replicates and compared against the transcriptome sequencing of an ethnicity matched population based control hyperplastic breast tissue.

Copy number variation (CNV) and gene expression were analyzed. Differentially expressed genes and 279 regions of CNV were found. 230 CNV regions overlapped the exons of annotated genes. While copy number change was significantly correlated to gene expression differences (2e-16), the effect was relatively small, explaining only 1.5% of the gene expression variation. Noting that while the correlation (0.3) and explained percent variance (0.093%) was higher in genes that were highly expressed (RPKM>0.1), there were still a large number of genes (2070) that were switched off or had significantly lower expression in tumor despite having high expression in normal tissue. Accordingly, expression of these genes appears to be disrupted.

### Example 2

### Integrated Analysis of Matched Normal and Tumor Whole Genome and Tumor Transcriptome Sequencing Data from Four African American Patients with Metastatic Chemo-Resistant TNBC

Although significant progress has been made towards treatment of hormone receptor (estrogen, progesterone) positive and HER2-neu positive breast tumors, women diagnosed with triple negative breast cancer (TNBC), or tumors that show no expression of these receptors, have few options and outcomes remain relatively poor. Furthermore, there is evidence that TNBC is more prevalent among young pre-menopausal women of recent west African descent, namely African American women. In many cases, TNBC exhibits phenotypic and genotypic characteristics similar to the basal-like or BRCA-associated breast cancers. These similarities have led to the development of therapies aimed at BRCA molecular mechanisms such as the class of PARP inhibitors that are showing promise in clinical trials with significant increases in time to progression and survival. However some TNBC have very different genotypic profiles and represent a heterogeneous class of aggressive breast cancers. In many cases, patients with an initial diagnosis of TNBC are cured with adjuvant chemotherapy and surgery, but roughly 30% of patients have rapid relapse of chemoresistant disease. In patients with metastatic chemoresistant TNBC, molecularly guided therapies can improve outcomes in patients as compared to conventional therapeutic decision methods. In this invention, a disease specific approach was undertaken to indentify molecularly targeted treatment options for patients with chemoresistant metastatic TNBC. To uncover targets within each patient's tumor, both whole genomes and transcriptomes were interrogated utilizing Next Generation Sequencing technologies (NGS). The results of deep whole genome and transcriptome sequencing of a metastatic tumor from a patient with chemoresistant TNBC may be used to discover therapeutically actionable targets for treatment to assist the patient treatment.

The integrated analysis of matched normal and tumor whole genome and tumor transcriptome sequencing data were from four African American patients with metastatic chemo-resistant TNBC for better understanding of the compendium of somatic events occurring in these tumors within this high-risk population.

Material and methods: Greater than 1 cm³ surgical fresh frozen tumor specimens were collected during surgery and quality assessed for tumor cellularity, necrosis, crush artifact, etc. A blood sample was also provided for the collection of constitutional genomic DNA. For differential expression analysis, a series of fresh frozen hyperplastic epithelial breast tissue specimens were obtained from, which were matched for age and ethnicity. RNA and DNA were extracted from biospecimens using the Qiagen All Prep kit (Germantown, MD). Table 2 provides information regarding patients and samples.

TABLE 2
Information regarding four African American TNBC patients and samples

| Participant | | Age at | Tumor | |
|---|---|---|---|---|
| Code | Ethnicity | Diagnosis | Cellularity | RNA RIN |
| TNBC-001 | African American | 52 | 95% | 9.8 |
| TNBC-003 | African American | 47 | 50% | 9.5 |
| TNBC-004 | African American | 54 | 80% | 9.0 |
| TNBC-007 | African American | 42 | 50% | 9.5 |

Next Generation Sequencing was performed using the SOLiD version 4.0 system. Patient matched tumor and germline genomes were sequenced to >20× coverage and analyzed using custom paired analysis tools to uncover somatic alterations. Tumor transcriptomes were sequenced (RNA-seq) to 30 million uniquely aligned reads using Life Technologies Bioscope (Applied Biosystems, Carlsbad, Calif.). For expression analysis, patient RNA-seq data are compared to data generated from ethnicity-matched population-based control hyperplastic breast tissue using EdgeR (Robinson et al, 2010).

On average, over 3 million germline variants were detected in the four African American patients. Somatic paired analysis has revealed point mutations, small indels, copy number alterations, and translocations, in known cancer genes, and has revealed potential novel genes as well. The germline variation review result also provides information on inherited breast cancer mutations. Among four African American patients sequenced, both unique and common genomic and transcriptomic perturbations were uncovered. In all patients, unique somatic genomic alterations are associated with upregulation of specific signaling pathways.

RNA-seq-based expression analysis has revealed a profile dominated by perturbations in cell cycle mitotic checkpoint defects. Furthermore, integrated analysis also provided key insights into the transcriptional consequences of a number of genomic alterations including exon skipping events and the discovery of potential fusion transcripts. Importantly, DNA and RNA changes have been validated by independent methods and technologies, such as expression microarray analysis, and Sanger sequencing.

Deep genome and transcriptome profiling of chemo-resistant TNBC has provided insights into events occurring in this cancer subtype which is difficult to treat. This invention provides the leveraging of these data to provide an opportunity for informed therapeutic options for intervention of this cancer subtype.

Paired Genome Sequencing

All NGS was carried out using the SOLiD™ version 4.0 mate-pair chemistry (Applied Biosystems by Life Technologies, Foster City, Calif.) following the manufacturer's instructions. Briefly, for each mate-pair sequencing library, 20micrograms (mg) high molecular weight genomic DNA was randomly fragmented using the Hydroshear (Applied Biosystems of Life Technologies, Foster City, Calif.) to produce libraries with of approximately 1.5 kilobasepairs (kb) inserts on average. Two independent mate-pair libraries were generated from the tumor DNA and two libraries from the patient's constitutional DNA. Emulsion PCR was used to amplify libraries, which were subsequently enriched using the EZBead emulsion PCR system (Applied Biosystems of Life Technologies, Foster City, Calif.) using the manufacturer's recommendations. Approximately 500,000,000 enriched templated sequencing beads were deposited on each SOLiD™ version 4.0 flowcell slide and sequenced to generate 50×50 bp mate-pair reads per templated bead.

TABLE 3
Genome sequencing run statistics for four African American TNBC

| | Total 50 × 50 bp | Total | | Number | | Transition/ |
|---|---|---|---|---|---|---|
| | Beads | Bases | | Germline | Percent | Transversion |
| Participant Code | Sequenced | Sequenced | Coverage | Variants | dbSNP | Ratio |
| TNBC-001 Normal | 2.2 billion | 95 Gb | 31X | 3.7 miilion | 79% | 2.0 |
| TNBC-001 Tumor | 1.9 billion | 89 Gb | 29X | — | | |
| TNBC-003 Normal | 2.3 billion | 100 Gb | 33X | 3.1 million | 80.08% | 2.0476 |
| TNBC-003 Tumor | 2.0 billion | 92 Gb | 31X | — | | |
| TNBC-004 Normal | 1.7 billion | 75 Gb | 25X | 3.0 million | 80.86% | 2.0536 |
| TNBC-004 Tumor | 1.8 billion | 82 Gb | 27X | — | | |
| TNBC-007 Normal | 2.4 billion | 106 Gb | 35X | 3.4 million | 80.73% | 2.08 |
| TNBC-007 Tumor | 2.6 billion | 119 Gb | 40X | — | | |

Transcriptome Sequencing.

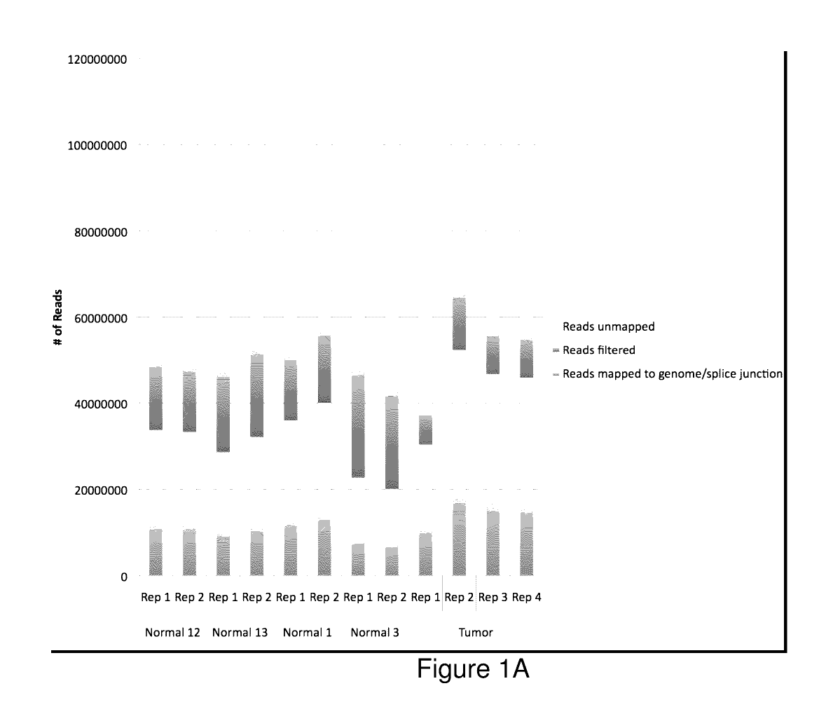

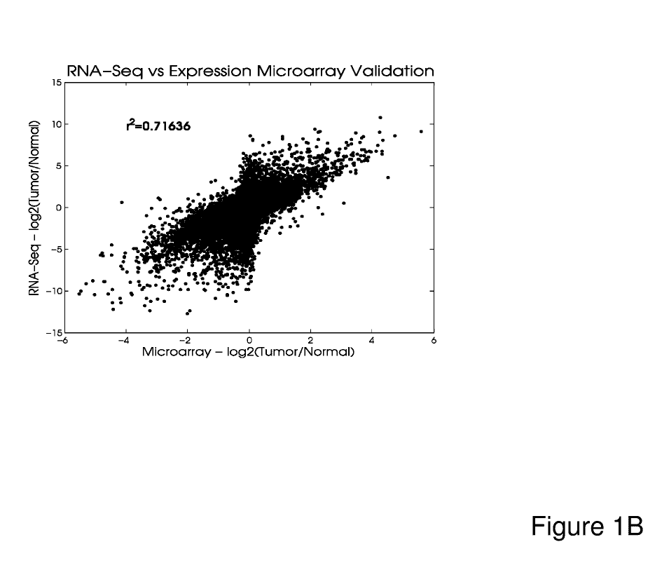

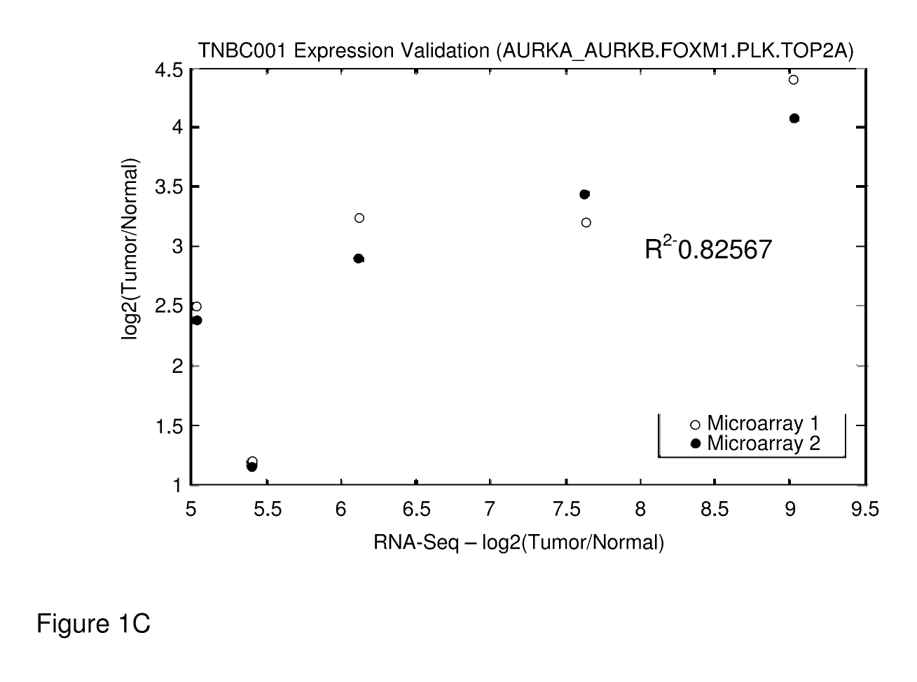

Whole transcriptome sequencing was carried out using the SOLiD™ Total RNA-Seq kit (Applied Biosystems of Life Technologies, Foster City, Calif.) using the manufacturer's recommendations. Briefly, 10 μg of total RNA was depleted of ribosomal RNA species using the Ribo-Minus kit (Invitrogen by Life Technologies, Carlsbad, Calif.) using the manufacturer's recommendations). To generate SOLiD RNA-Seq fragment libraries, 200-500 nanograms (ng) of ribosomal RNA depleted total RNA was fragmented by sonication. Size selected fragments were ligated to SOLiD™ RNA-Seq specific sequencing adaptors and a reverse transcription was carried out to generate cDNA. These cDNA molecules are further size selected and amplified. An emulsion PCR was carried out and ˜100,000,000 enriched templated sequencing beads were deposited for sequencing for each RNA-Seq library. FIG. 1 depicts A. Histogram illustrating mapping statistics for RNA-seq experiments for TNBC-001 and normal breast specimens. B. Scatter plot illustrating correlation coefficients for RNA-seq versus microarray-based gene expression results for TNBC-001. C. Correlation coefficients for specific genes measured by RNA-seq versus microarray-based gene expression differences for TNBC-001 versus normal breast samples.

Paired Somatic Single Nucleotide Variant Detection

For paired somatic single nucleotide detection, SolSNP was utilized to detect single nucleotide variants. However, variants are called conservatively in germlines in order to reduce false positives, and variants are called liberally in tumors in order to reduce false negatives. A custom script was then utilized to compare variants to search for tumor specific changes using an algorithm, which provides a Phred like statistical score for likelihood of a somatic variant being truly tumor specific.

Paired Somatic Copy Number Analysis

For copy number analysis, a custom tool was developed based on a sliding window comparison of coverage for tumor/normal. These analyses utilized a 1 kb sliding window, with coverage differences output as log2 ratios and plotted against the genome to identify regions of gain or loss in the tumor.

Paired Somatic Insertion/Deletion Detection

For detecting somatic indels a two step strategy was employed. In the first step we removed from the tumor sample bam, reads whose insert size lay outside a 500 bp-5000 bp interval for SOLiD. Genome Analysis Toolkit (GATK) is then used to generate a list of potential small indels from this bam. A customized Perl script, which uses the Bio-SamTools library from BioPerl 9Stajich et al Genome Res. 2002 October; 12(10):1611-8), takes these indel positions and for each of the indels looks at the region in the normal sample consisting of 5 bp upstream from the start and 5 bp downstream from the end of the indel. An indel is determined to be somatic only if there was no indel detected in the region under consideration.

Paired Somatic Translocation Detection

A series of customized Perl scripts were employed in the detection of translocation. These scripts used SAMtools (McKenna A. et al Genome Res. 2010 September; 20(9):1297-303) internally to access the bam files. The algorithm consists of two steps. The first is to detect potential translocation in both tumor and normal samples. The second is a comparison of potential translocations in tumor to those detected in the normal sample to weed out potential false positives. The detection of a potential translocation was an exercise in outlier detection. A sliding window of 2 kbp was focused and the discordant reads were counted, whose mates align on a different chromosome. A 2 kbp window size was used, as it is close to the mean of the estimated insert size distribution, and gave the best resolution for the detection of an interchromosomal translocation using 1.5 kb mate pair libraries. For each window we chose the highest hit to be the chromosome to which mates of most of the discordant reads mapped. For purposes of brevity, the subset of discordant reads whose mate maps to the highest hit in the window as the hit discordant reads was called. The ratios of the hit discordant reads were compared to the total aligned reads, across all the windows to detect potential outliers. Outlier detection was performed under the assumption that the distribution, of the proportion of hit discordant reads in a 2 kb window aggregates across the chromosome, and will follow a normal distribution. The mean of this distribution was then computed and a cutoff of 3 standard deviations was chosen. The window with a proportion of hit discordant reads, higher than this cutoff contains the region of potential translocation. The actual region of translocation was then determined by the span of the hit discordant reads in the window. For somatic translocations, the normal and the tumor sample were called separately and regions of overlap were eliminated. These regions were further inspected visually to reduce false positives and arrive at the most confident list.

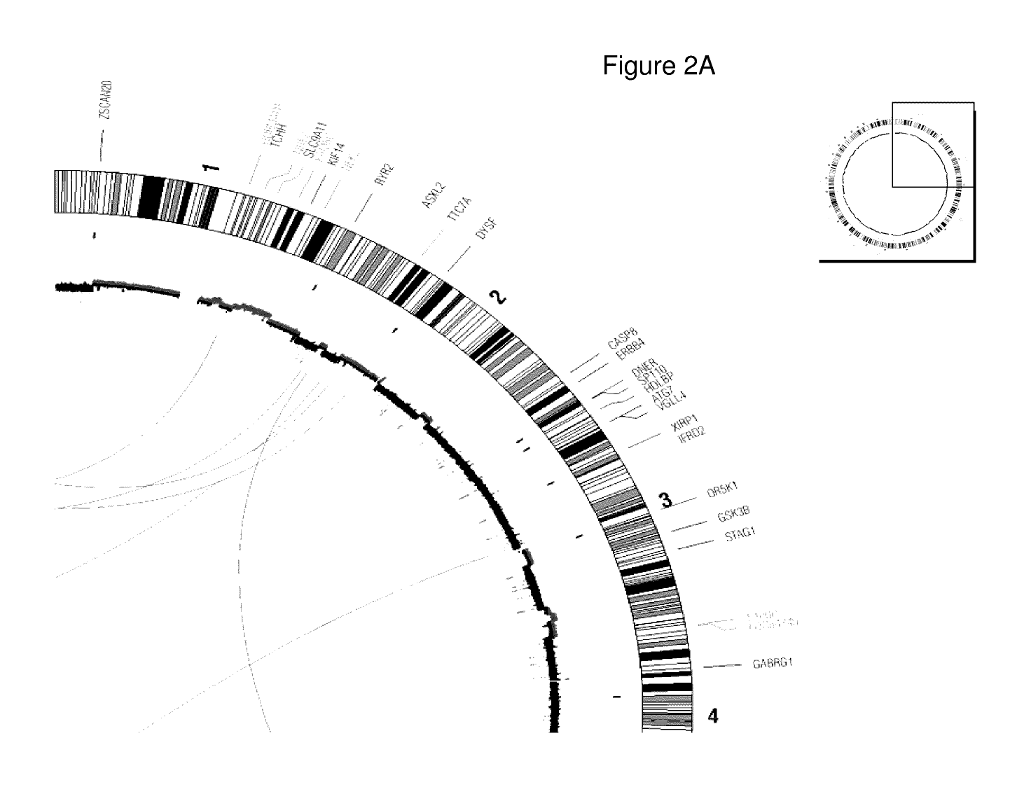

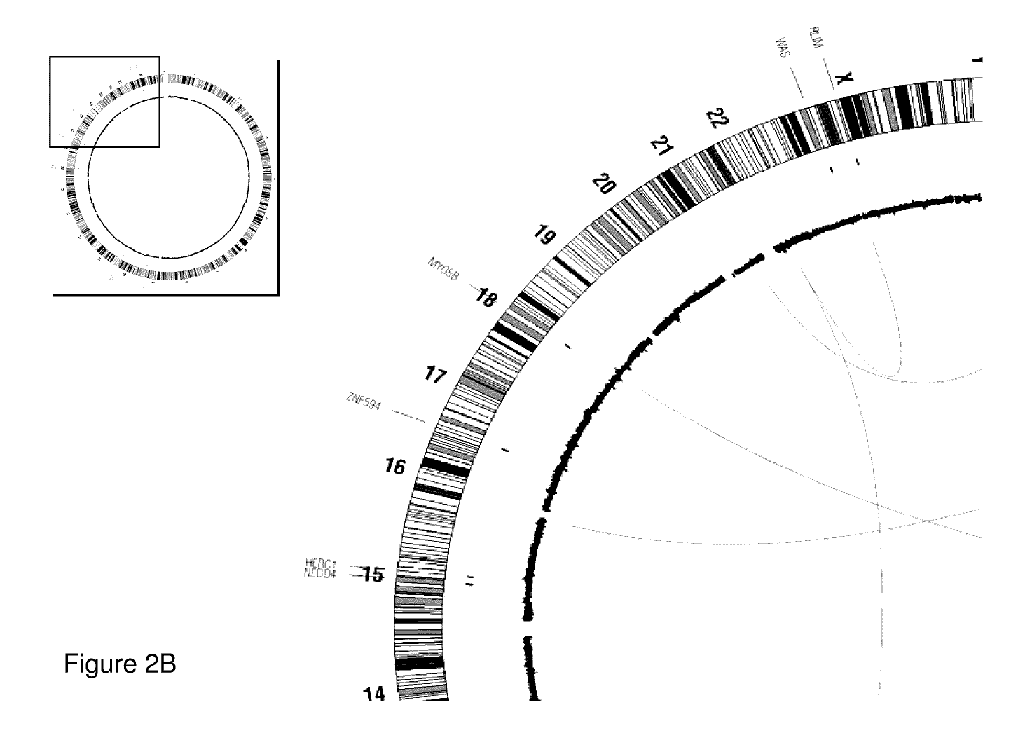

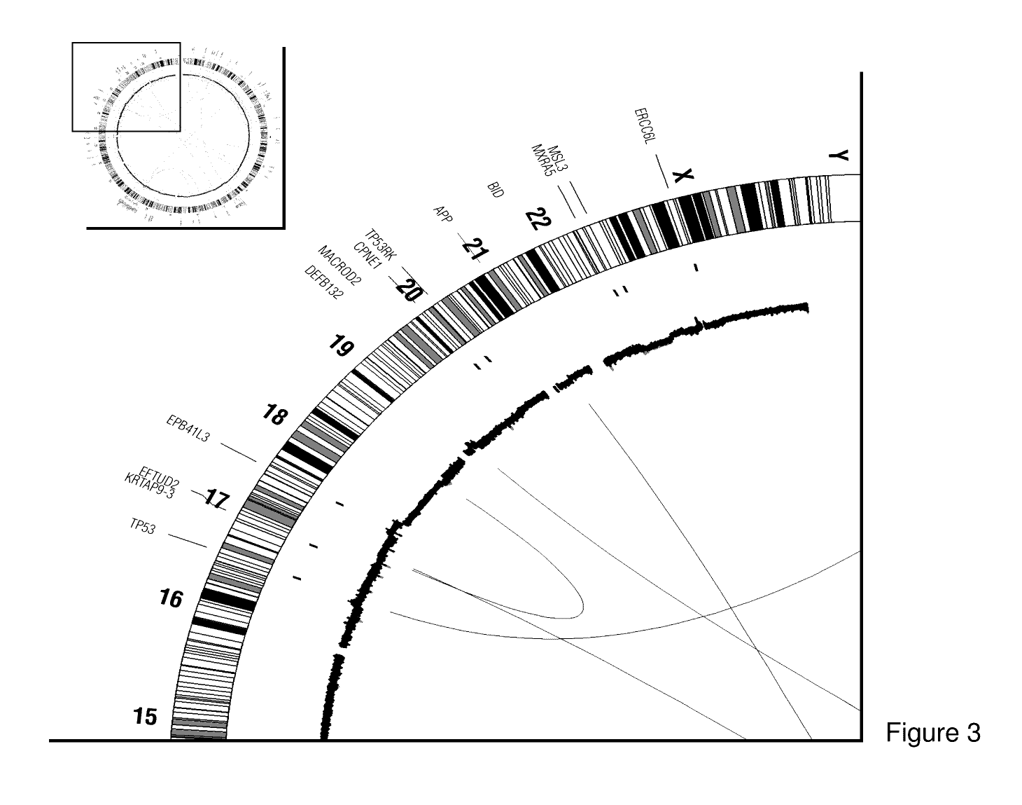

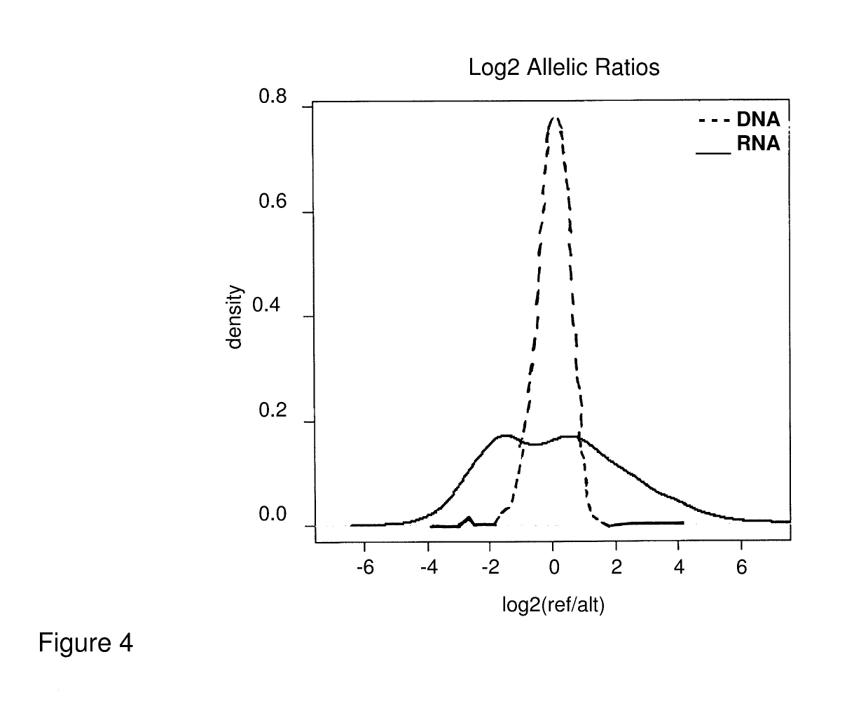

FIG. 2 presents a Circos plot illustrating somatic events detected in TNBC-001 with gene affected listed in Table 4 (FIG. 2A) and somatic events detected in TNBC-003 with gene affected listed in Table 5 (FIG. 2B). FIG. 3 depicts the Circos plot illustrating somatic events occurring in TNBC-004, with gene affected listed in Table 6.

TABLE 4
Genes with somatic events in FIG. 2A—TNBC-001

| | | | | | |
|---|---|---|---|---|---|
| | | UniProtKB/ | | | UniProtKB/ |
| | | Swiss- | | | Swiss- |
| | | Prot ID | Gene | | Prot ID |
| Gene Name | Protein Name | Number | Name | Protein Name | Number |

Somatic point mutation

| | | | | | |
|---|---|---|---|---|---|
| ZSCAN20 | Zinc finger and SCAN | P17040 | PBLD | Phenazine | P30039 |
| | domain-containing | | | biosynthesis-like | |
| | protein | | | domain-containing | |
| | | | | protein | |
| KIF14 | Kinesin-like protein | Q15058 | OR5AR1 | Olfactory receptor | Q8NGP9 |
| | KIF14 | | | 5AR1 | |
| RYR2 | Ryanodine receptor 2 | Q92736 | SLC2A14 | Solute carrier | Q8TDB8 |
| | | | | family 2, | |
| | | | | facilitated glucose | |
| | | | | transporter | |
| | | | | member 14 | |
| DYSF | Dysferlin | O75923 | RAPGEF3 | Rap guanine | O95398 |
| | | | | nucleotide | |
| | | | | exchange factor 3 | |
| DNER | Delta and Notch-like | Q8NFT8 | PTPRB | Receptor-type | P23467 |
| | epidermal growth | | | tyrosine-protein | |
| | factor-related receptor | | | phosphatase beta | |
| SP110 | Sp110 nuclear body | Q9HB58 | C12orf30 | N-alpha- | Q14CX7 |
| | protein | | | acetyltransferase | |
| | | | | 25, NatB auxiliary | |
| | | | | subunit | |
| HDLBP | Vigilin | Q00341 | RB1 | Retinoblastoma- | P06400 |
| | | | | associated protein | |
| XIRP1 | Xin actin-binding | Q702N8 | FGF14 | Fibroblast growth | Q92915 |
| | repeat-containing | | | factor 14 | |
| | protein 1 | | | | |
| OR5K1 | Olfactory receptor 5K1 | Q8NHB7 | PLEKHC1 | Fermitin family | Q96AC1 |
| | | | | homolog 2 | |
| NDST3 | Bifunctional heparan | O95803 | DACT1 | Dapper homolog 1 | Q9NYF0 |
| | sulfate | | | | |
| | N-deacetylase/ | | | | |
| | N-sulfotransferase 3 | | | | |
| PCDHB8 | Protocadherin beta-8 | Q9UN66 | KIAA1370 | Uncharacterized | Q32MH5 |
| | | | | protein KIAA1370 | |
| IL12B | Interleukin-12 subunit | P29460 | ABCC12 | Multidrug | Q96J65 |
| | beta | | | resistance- | |
| | | | | associated protein | |
| | | | | 9 | |
| GNA12 | Guanine nucleotide- | Q03113 | MYOM1 | Myomesin-1 | P52179 |
| | binding protein subunit | | | | |
| | alpha-12 | | | | |
| ABCB1 | Multidrug resistance | P08183 | SLC7A10 | Asc-type amino | Q9NS82 |
| | protein 1 | | | acid transporter 1 | |
| LMOD2 | Leiomodin-2 | Q6P5Q4 | ZNF233 | Zinc finger protein | A6NK53 |
| | | | | 233 | |
| C8orf58 | Uncharacterized | Q8NAV2 | KLK13 | Kallikrein-13 | Q9UKR3 |
| | protein C8orf58 | | | | |
| PTPLAD2 | 3-hydroxyacyl-CoA | Q5VWC8 | EYA2 | Eyes absent | O00167 |
| | dehydratase 4 | | | homolog 2 | |
| FBP1 | Fructose-1,6- | P09467 | SERPIND1 | Heparin cofactor 2 | P05546 |
| | bisphosphatase 1 | | | | |
| KIAA0368 | Proteasome-associated | Q5VYK3 | CYTSA | Cytospin-A | Q69YQ0 |
| | protein ECM29 | | | | |
| | homolog | | | | |
| GDI2 | Rab GDP dissociation | P50395 | SEC14L2 | SEC14-like | O76054 |
| | inhibitor beta | | | protein 2 | |

Somatic indel

| | | | | | |
|---|---|---|---|---|---|
| TCHH | Trichohyalin | Q07283 | CHRNA10 | Neuronal | Q9GZZ6 |
| | | | | acetylcholine | |
| | | | | receptor subunit | |
| | | | | alpha-10 | |
| ASXL2 | Putative Polycomb | Q76L83 | SMPD1 | Sphingomyelin | P17405 |
| | group protein ASXL2 | | | phosphodiesterase | |
| IFRD2 | Interferon-related | Q12894 | HNF1A | Hepatocyte | P20823 |
| | developmental | | | nuclear factor | |
| | regulator 2 | | | 1-alpha | |
| TERT | Tert protein | A0JNY9 | PTEN | Phosphatidylinositol- | P60484 |
| | | | | 3,4,5-trisphosphate | |
| | | | | 3-phosphatase and | |
| | | | | dual-specificity | |
| | | | | protein phosphatase | |
| | | | | PTEN | |
| NEUROG1 | Neurogenin-1 | Q92886 | ELMO3 | Engulfment and | Q96BJ8 |
| | | | | cell motility | |
| | | | | protein 3 | |
| DNAI1 | Dynein intermediate | Q9UI46 | DLGAP4 | Disks large- | Q9Y2H0 |
| | chain 1, axonemal | | | associated | |
| | | | | protein 4 | |
| AMBP | Protein AMBP | P02760 | TFDP3 | Transcription | Q5H9I0 |
| | | | | factor Dp family | |
| | | | | member 3 | |

Copy number amplification

| | | | | | |
|---|---|---|---|---|---|
| HORMAD1 | HORMA domain- | Q86X24 | CA9 | Carbonic | Q16790 |
| | containing protein 1 | | | anhydrase 9 | |
| NUF2 | Kinetochore protein | Q9BZD4 | MUC5B | Mucin-5B | Q9HC84 |
| | Nuf2 | | | | |
| FAM5C | Protein FAM5C | Q76B58 | LRTM2 | Leucine-rich | Q8N967 |
| | | | | repeat and | |
| | | | | transmembrane | |
| | | | | domain-containing | |
| | | | | protein 2 | |
| NEK2 | Serine/threonine- | P51955 | FOXM1 | Forkhead box | Q08050 |
| | protein kinase Nek2 | | | protein M1 | |
| S100P | S100P-binding protein | Q96BU1 | NOXO1 | NADPH oxidase | Q8NFA2 |
| | | | | organizer 1 | |
| LOC84740 | | | UHRF1 | E3 ubiquitin- | Q96T88 |
| | | | | protein ligase | |
| | | | | UHRF1 | |
| COL9A1 | Collagen alpha-1(IX) | P20849 | NLRP9 | NACHT, LRR and | Q7RTR0 |
| | chain | | | PYD domains- | |
| | | | | containing protein | |
| | | | | 9 | |
| PRDM13 | PR domain zinc finger | Q9H4Q3 | UBE2C | Ubiquitin- | O00762 |
| | protein 13 | | | conjugating | |
| | | | | enzyme E2 C | |
| ULBP2 | NKG2D ligand 2 | Q9BZM5 | KIF4A | Chromosome- | O95239 |
| | | | | associated kinesin | |
| | | | | KIF4A | |
| DMRT1 | Doublesex- and mab-3- | Q3LH63 | PCDH11X | Protocadherin-11 | Q9BZA7 |
| | related transcription | | | X-linked | |
| | factor 1 | | | | |

Copy number deletion

| | | | | | |
|---|---|---|---|---|---|
| HSPB7 | Heat shock protein | Q9UBY9 | AQP7 | Aquaporin-7 | O14520 |
| | beta-7 | | | | |
| DARC | DARC protein | Q4VBN9 | AKR1C2 | Aldo-keto | P52895 |
| | | | | reductase family 1 | |
| | | | | member C2 | |
| ADIPOQ | Progestin and adipoQ | Q6TCH4 | RBP4 | Retinol-binding | Q00724 |
| | receptor family | | | protein 4 | |
| | member 6 | | | | |
| MMRN1 | Multimerin-1 | Q13201 | ADAMTS15 | A disintegrin and | Q8TE58 |
| | | | | metalloproteinase | |
| | | | | with | |
| | | | | thrombospondin | |
| | | | | motifs 15 | |
| IRX1 | Iroquois-class | P78414 | STAB2 | Stabilin-2 | Q8WWQ8 |
| | homeodomain protein | | | | |
| | IRX-1 | | | | |
| LEP | Lens epithelial cell | Q9Y5L5 | CD300LF | CMRF35-like | Q8TDQ1 |
| | protein LEP503 | | | molecule 1 | |
| FABP4 | Fatty acid-binding | P15090 | GFAP | Glial fibrillary | P14136 |
| | protein, adipocyte | | | acidic protein | |

Translocation

| | | | | | |
|---|---|---|---|---|---|
| SLC9A11 | Sodium/hydrogen | Q5TAH2 | EXOC4 | Exocyst complex | Q96A65 |
| (with) | exchanger 11 | | (with) | component 4 | |
| NCOA6 | Nuclear receptor | Q14686 | BTBD1 | BTB/POZ | Q9H0C5 |
| | coactivator 6 | | | domain-containing | |
| | | | | protein 1 | |
| TTC7A | Tetratricopeptide | Q9ULT0 | FRMD4A | FERM domain- | Q9P2Q2 |
| (with) | repeat protein 7A | | (with) | containing protein | |
| SPRED1 | | | OGT | 4A | |
| | Sprouty-related, EVH1 | Q7Z699 | | Ogt protein | Q8C676 |
| | domain-containing | | | | |
| | protein 1 | | | | |
| SNX9 | Sorting nexin-9 | Q91VH2 | | | |
| (with) | Sequestosome-1 | Q13501 | | | |
| EBI3 | | | | | |

Other

| | | | | | |
|---|---|---|---|---|---|
| ERICH1 | Glutamate-rich protein | Q86X53 | GABRG1 | Gamma- | Q8N1C3 |
| | 1 | | | aminobutyric acid | |
| | | | | receptor subunit | |
| | | | | gamma-1 | |
| ACVR1B | Activin receptor | P36896 | LRBA | olysaccharide- | P50851 |
| | type-1B | | | responsive and | |
| | | | | beige-like anchor | |
| | | | | protein | |
| C16orf70 | UPF0183 protein | Q9BSU1 | SPOCK3 | Testican-3 | Q9BQ16 |
| | C16orf70 | | | | |
| CDH26 | Cadherin-like protein | Q8IXH8 | CTNNA1 | Catenin alpha-1 | P35221 |
| | 26 | | | | |
| IRAK1 | Interleukin-1 receptor- | P51617 | PCDHA8 | Protocadherin | Q9Y5H6 |
| | associated kinase 1 | | | alpha-8 | |
| CASPB | Caspase | Q504J1 | ASTN2 | Astrotactin-2 | O75129 |
| ERBB4 | Receptor tyrosine- | Q15303 | C10orf79 | WD repeat- | Q8NDM7 |
| | protein kinase erbB-4 | | | containing protein | |
| | | | | 96 | |
| ATG7 | Ubiquitin-like | O95352 | FLT3 | Receptor-type | P36888 |
| | modifier-activating | | | tyrosine-protein | |
| | enzyme ATG7 | | | kinase FLT3 | |
| VGLL4 | Transcription cofactor | Q14135 | POTEB | POTE ankyrin | Q6S5H4 |
| | vestigial-like protein 4 | | | domain family | |
| | | | | member B | |
| GSK3B | Glycogen synthase | P49841 | OR4M2 | Olfactory receptor | Q8NGB6 |
| | kinase-3 beta | | | 4M2 | |
| STAG1 | Cohesin subunit SA-1 | Q8WVM7 | | | |

TABLE 5
Genes with somatic events in FIG. 2B—TNBC-003

| | | | | | |
|---|---|---|---|---|---|
| | | UniProtKB/ | | | UniProtKB/ |
| | | Swiss- | | | Swiss- |
| Gene | | Prot ID | Gene | | Prot ID |
| Name | Protein Name | Number | Name | Protein Name | Number |

Somatic point mutation

| | | | | | |
|---|---|---|---|---|---|
| GPAM | Glycerol-3-phosphate | Q9HCL2 | ZNF594 | Zinc finger protein | Q96JF6 |
| | acyltransferase 1, | | | 594 | |
| | mitochondrial | | | | |
| INPP5F | Phosphatidylinositide | Q9Y2H2 | MYO5B | Myosin-Vb | Q9ULV0 |
| | phosphatase SAC2 | | | | |
| NEDD4 | E3 ubiquitin-protein | P46934 | WAS | WAS protein | Q9NQA3 |
| | ligase NEDD4 | | | family homolog | |
| HERC1 | Probable E3 ubiquitin- | Q15751 | RLIM | E3 ubiquitin- | Q9NVW2 |
| | protein ligase HERC1 | | | protein ligase | |
| | | | | RLIM | |

Other

| | | | | | |
|---|---|---|---|---|---|
| TLR4 | TLR4 interactor with | Q7L0X0 | C5 | Complement C5 | P01031 |
| | leucine rich repeats | | | | |

TABLE 6
Genes with somatic events in FIG. 3—TNBC-005

| | | | | | |
|---|---|---|---|---|---|
| | | UniProtKB/ | | | UniProtKB/ |
| | | Swiss- | | | Swiss- |
| Gene | | Prot ID | Gene | | Prot ID |
| Name | Protein Name | Number | Name | Protein Name | Number |

Somatic point mutation

| | | | | | |
|---|---|---|---|---|---|
| MAN1A2 | Mannosyl- | O60476 | ANKRD26 | Ankyrin repeat | Q9UPS8 |
| | oligosaccharide 1,2- | | | domain-containing | |
| | alpha-mannosidase IB | | | protein 26 | |
| GON4L | GON-4-like protein | Q3T8J9 | ANKRD30A | Ankyrin repeat | Q9BXX3 |
| | | | | domain-containing | |
| | | | | protein 30A | |
| POGK | Pogo transposable | Q9P215 | FAM13C | Protein FAM13C | Q8NE31 |
| | element with KRAB | | | | |
| | domain | | | | |
| KIAA1614 | Uncharacterized | Q5VZ46 | AP001998.1 | | |
| | protein KIAA1614 | | | | |
| SLC45A3 | Solute carrier family | Q96JT2 | GAB2 | GRB2-associated- | Q9UQC2 |
| | 45 member 3 | | | binding protein 2 | |
| THSD7B | Thrombospondin type- | Q9C0I4 | BAZ1A | Bromodomain | Q9NRL2 |
| | 1 domain-containing | | | adjacent to zinc | |
| | Protein 7B | | | finger domain | |
| | | | | protein 1A | |
| AC109825.1 | | | B019439.1 | | |
| FOXP1 | Forkhead box protein | Q9H334 | TP53 | TP53-target gene 5 | Q9Y2B4 |
| | P1 | | | protein | |
| KCNMB2 | Calcium-activated | Q9Y691 | EFTUD2 | 116 kDa U5 small | Q15029 |
| | potassium channel | | | nuclear | |
| | subunit beta-2 | | | ribonucleoprotein | |
| | | | | component | |
| KIAA1244 | Brefeldin A-inhibited | Q5TH69 | EPB41L3 | Band 4.1-like | Q9Y2J2 |
| | guanine nucleotide- | | | protein 3 | |
| | exchange protein 3 | | | | |
| IGF2R | Cation-independent | P11717 | CPNE1 | Copine-1 | Q99829 |
| | mannose-6-phosphate | | | | |
| | receptor | | | | |
| RBAK | RB-associated KRAB | Q9NYW8 | TP53RK | TP53-regulating | Q96S44 |
| | zinc finger protein | | | kinase | |
| DGKI | Diacylglycerol kinase | O75912 | MXRA5 | Matrix- | Q9NR99 |
| | iota | | | remodeling- | |
| | | | | associated | |
| | | | | protein 5 | |
| ADHFE1 | Hydroxyacid-oxoacid | Q8IWW8 | MSL3 | Male-specific | Q8N5Y2 |
| | transhydrogenase, | | | lethal 3 homolog | |
| | mitochondrial | | | | |
| ERCC6L | DNA excision repair | Q2NKX8 | | | |
| | protein ERCC-6-like | | | | |

somatic indel

| | | | | | |
|---|---|---|---|---|---|
| CDCP2 | CUB domain- | Q5VXM1 | DEFB132 | Beta-defensin 132 | Q7Z7B7 |
| | containing protein 2 | | | | |

Copy number amplification

| | | | | | |
|---|---|---|---|---|---|
| DCDC1 | Doublecortin domain- | P59894 | WT1 | PRKC apoptosis | Q96IZ0 |
| | containing protein 1 | | | WT1 regulator | |
| | | | | protein | |
| DNAJC24 | DnaJ homolog | Q6P3W2 | | | |
| | subfamily C member | | | | |
| | 24 | | | | |

Copy number deletion

| | | | | | |
|---|---|---|---|---|---|
| C2orf34 | Calmodulin-lysine | Q7Z624 | SYTL3 | Synaptotagmin- | Q4VX76 |
| | N-methyltransferase | | | like protein 3 | |
| ERBB4 | Receptor tyrosine- | Q15303 | c9orf93 | Uncharacterized | Q6TFL3 |
| | protein kinase erbB-4 | | | protein C9orf93 | |
| RPL15 | 60S ribosomal protein | P61313 | IFITM5 | Interferon-induced | A6NNB3 |
| | L15 | | | transmembrane | |
| | | | | protein 5 | |
| NDUFAF3 | DH dehydrogenase | Q9BU61 | SYT8 | Synaptotagmin-8 | Q8NBV8 |
| | [ubiquinone] 1 alpha | | | | |
| | subcomplex assembly | | | | |
| | factor 3 | | | | |
| LAMB2 | Laminin subunit beta-2 | P55268 | TNNI2 | Troponin I, fast | P48788 |
| | | | | skeletal muscle | |
| BSN | Probable DNA repair | E8VIW0 | CD81 | CD81 antigen | P60033 |
| | protein BSn5_18740 | | | | |
| KCTD8 | BTB/POZ domain- | Q6ZWB6 | KCNQ1 | KCNQ1 | Q9H478 |
| | containing protein | | | downstream | |
| | KCTD8 | | | neighbor protein | |
| TBCKL | TBC domain- | Q8TEA7 | SLC22A18 | Solute carrier | Q96BI1 |
| | containing protein | | | family 22 member | |
| | kinase-like protein | | | 18 | |
| NDST3 | Bifunctional heparan | O95803 | OR51A2 | Olfactory receptor | Q8NGJ7 |
| | sulfate N- | | | 51A2 | |
| | deacetylase/N- | | | | |
| | sulfotransferase 3 | | | | |
| MAP1B | Microtubule-associated | P46821 | ADM | ADM | P35318 |
| | protein 1B | | | | |
| LNPEP | Leucyl-cystinyl | Q9UIQ6 | PCDH9 | Protocadherin-9 | Q9HC56 |
| | aminopeptidase | | | | |
| FSTL4 | Follistatin-related | Q6MZW2 | TNFSF13B | Tumor necrosis | Q9Y275 |
| | protein 4 | | | factor ligand | |
| | | | | superfamily | |
| | | | | member 13B | |
| ARID1B | AT-rich interactive | Q8NFD5 | ACOT1 | Acyl-coenzyme A | Q86TX2 |
| | domain-containing | | | thioesterase 1 | |
| | protein 1B | | | | |
| APBA2 | | | MACROD2 | MACRO domain- | A1Z1Q3 |
| | | | | containing protein | |
| | | | | 2 | |
| KRTAP9-3 | Keratin-associated | Q9BYQ3 | BID | BH3-interacting | P55957 |
| | protein 9-3 | | | domain death | |
| | | | | agonist | |

Translocation

| | | | | | |
|---|---|---|---|---|---|
| STIL | SCL-interrupting | Q15468 | WBSCR17 | Putative | Q61524 |
| (with) | locus protein | | (with) | polypeptide N- | |
| KLF11 | | | APP | acetylgalactosamin | |
| | | | | yltransferase-like | |
| | | | | protein 3 | |
| | Krueppel-like factor | O14901 | | Amyloid beta A4 | P05067 |
| | 11 | | | protein | |

Other

| | | | | | |
|---|---|---|---|---|---|
| PGCP | Plasma glutamate | Q9Y646 | PEX16 | Peroxisomal | Q9Y5Y5 |
| | carboxypeptidase | | | membrane protein | |
| | | | | PEX16 | |

Germline Variant Detection

To identify SNPs and variation in patient germline DNA samples, SolSNP was utilized, which is an individual sample variant detector (classifier) implemented in Java. The variant calling was based on a modified Kolmogorov-Smirnov like statistic. The algorithm is non-parametric and makes no assumptions on the nature of the data. It compared the discrete sampled distribution, the pileup on each strand, to the expected distributions (according to ploidy). An important aspect of SolSNP that reduces overcalling inherent to the K-S statistic algorithm is that filters are included to reduce false positive rates, among of which is that both strands must provide evidence for the variation.

Transcriptome Analysis

SOLiD™ BioScope™ Whole Transcriptome Analysis (WTA) pipeline for single reads was used to align the reads, count aligned reads per exon, and calculate per base coverage. Reads were aligned to three references: whole human genome build 36, splice junction reference derived from refSeq annotations and a filter reference which contains rRNA, tRNA, single-base-repeat (e.g. poly-A and T) and adapter sequences. Aligned reads from the alignments were merged into a final mapped reads BAM file. Any reads aligned to the filter genome were not included in the mapped reads BAM. Filtered reads were put in a filtered reads BAM and unmapped reads were put in a separate unmapped reads BAM file. WTA pipeline CountTags module provided normalize RPKM (Reads Per Kilobase of exon sequence, per Million reads) values along with read counts per exon. Bam2Wig module calculated coverage per base and produces WIG files containing the coverage information. BioScope 1.2.1 version was used with all default WTA settings.

Differential Expression Analysis

EdgeR, a Bioconductor package specifically for the differential expression analysis on the counts per gene from the tumor and normal samples, was used. Gene counts were normalized using EdgeR's TMM (trimmed mean M(=log fold-change gene expression)) algorithm. TMM attempts to adjust for unequal transcriptome sizes between samples by calculating an “effective” library size that is used to normalize the counts. The “effective” library size was calculated by multiplying or dividing the original library size by an offset. EdgeR's differential expression analysis was based on the assumption that the biological replicates follow a negative binomial distribution and technical replicates followed a Poisson distribution. For this study, the normal samples' technical replicates were merged and the analysis was done on the biological replicates. Gene expression was corrected using a moderated binomial dispersion correction, and then an exact test (similar to Fisher's exact test) was used to assess differential expression (Table 7). EdgeR's exact test produced a table of log concentrations, log fold-changes, p-values and false discovery rate (FDR).

TABLE 7
Differential expression data for several genes of interest

| | TNBC001 | TNBC003 | TNBC003 | TNBC004 | TNBC006 | Average Expression |
|---|---|---|---|---|---|---|
| | (AA) | (AA) | (AA) | (AA) | (AA) | in AA |
| Gene | LogFC | LogFC | LogFC | LogFC | LogFC | LogFC |
| FOXM1 | 9.030038261 | 7.936795268 | 7.936795268 | 7.818989515 | 1.74435731 | 6.893395124 |
| AURKA | 5.023398904 | 3.735583244 | 3.735583244 | 4.782064914 | 4.316994603 | 4.318724982 |
| AURKB | 7.628388957 | 5.423950537 | 5.423950537 | 6.260125132 | 2.531594525 | 5.453601938 |
| PLK1 | 5.398756206 | 5.207047596 | 5.207047596 | 5.636556518 | 4.03842049 | 5.097565681 |
| PTEN | −2.562997268 | −1.086504562 | −1.086504562 | −2.009955576 | −1.122789702 | −1.573750334 |
| IN PP4B | −2.806133878 | −0.592688104 | −0.592688104 | −3.087975478 | −2.44790344 | −1.905477801 |
| ERBB4 | −5.134278006 | −5.981572808 | −5.981572808 | −8.575178051 | −7.5329032 | −6.641100975 |

Five metastatic chemo-resistant TNBC normal/tumor pairs were completed in both whole genome and transcriptome sequencing. Quality control and assessment suggests high quality genome and transcriptome data. Initial analyses revealed several novel and known events involved in TNBC (Table 8). Somatic alterations at the ERBB4 locus may be common in metastatic chemo-resistant TNBC among African American women. ERBB4 intronic 4 kb homozygous deletion results in loss of expression in TNBC-001. In addition there is a significant intrachromosomal 21Mb deletion on chromosome 2 in mate pairs, which breaks ERBB4 and ATG16L1 in TNBC-007. Further analysis uncovered new insights into the genes and molecular pathways commonly perturbed in metastatic chemo-resistant TNBC among African American women.

TABLE 8
Genes commonly perturbed in metastatic chemo-resistant TNBC

| | | UniProtKB/ | | |
|---|---|---|---|---|
| Gene | | Swiss-Prot | Somatic | Differential |
| Name | Protein Name | ID Number | Alteration | Expression |
| FOXM1 | Forkhead box | Q08050 | | Up |
| | protein M1 | | | |
| AURKA | Aurora kinase A | O14965 | | Up |
| AURKB | Aurora kinase B | 96GD4 | | Up |
| PLK1 | Serine/threonine- | P53350 | | Up |
| | protein kinase | | | |
| | PLK1 | | | |
| PTEN | Mutated in | P60484 | yes | Down |
| | multiple | | | |
| | advanced cancers | | | |
| | 1 | | | |
| INPP4B | Type II inositol- | O15327 | | Down |
| | 3,4-bisphosphate | | | |
| | 4-phosphatase | | | |
| ERBB4 | Receptor | Q15303 | yes | Down |
| | tyrosine-protein | | | |
| | kinase erbB-4 | | | |
| ATG19L1 | | | yes | |

The DNA and RNA of TNBC samples were sequenced in order to identify relationships between somatic changes and expression. Integrated analysis of matched normal and tumor whole genome data, along with tumor transcriptome sequencing data were obtained from multiple samples with metastatic chemo-resistant TNBC. Two independent 1.5 kb Mate-pair libraries were generated for both tumor and germ-line derived genomic DNA and sequenced using SOLiD™ 4.0 paired 50mers to a target of 30x depth.

The tumor transcriptome was sequenced on four replicates and compared to transcriptome sequencing from ethnicity-matched population-based control hyperplastic breast tissue. Genome analysis was performed using multiple aligners and variant callers. Transcriptome alignment was performed using Bioscope™ pipeline, and differential expression analysis was performed using EdgeR (Robinson et al, 2010) and DESeq (Anders, 2010). Germline and somatic variants were annotated by integrative analysis with differential expression results. Several striking examples of intronic events correlating with either altered splicing or differential expression were observed in genes suggesting that transcriptomic data may have high value in interpreting somatic events that fall outside of coding regions. Final integration of data was validated through knowledge mining and convergence of somatic events and expression.

For RNA sequencing, normal breast tissue was collected from two human populations to match and/or compare to gene expression in samples with tumors. The specialized tumor-normal variant call pipeline included a somatic ‘copy number variant’ (CNV) caller, a germline SNP caller, two annotated somatic ‘single nucleotide variant’ (SNV, i.e., single nucleotides that differ from the standard human reference sequence) callers, a somatic translocation tool and an annotated somatic indel caller based on GATK.

Somatic mutation calls by the pipeline were manually evaluated using Integrative Genomics Viewer (IGV). By viewing the normal and tumor BAM files on the same slide it is possible to look at the distribution and enrichment of patterns to eliminate false positives that occur due to complexity of the human genome. Allelic ratios calculated from tumor RNA libraries showed increased variation compared to DNA allelic ratios suggesting the extent of functionally important regulatory variation.

Following two stringency criteria, it was determined that 105 and 2167 genes (enrichments summarized in Table 9) respectively, that are differentially expressed in 7 TNBC samples when compared to two populations of normal RNAs (from African American and Caucasian American samples). These enriched genes usually clustered together, and the enriched categories of gene ontology, pathways and protein domains of most differentially expressed genes are shown in Table 9:

TABLE 9
enriched categories of gene ontology, pathways and protein domains of most differentially expressed genes

| Category | Term | Count | % | PValue |
|---|---|---|---|---|
| GOTERM_BP_FAT | GO: 0008544~epidermis development | 60 | 2.911208 | 9.35E−18 |
| (Gene Oncology) | | | | |
| GOTERM_BP_FAT | GO: 0007398~ectoderm development | 62 | 3.008248 | 2.98E−17 |
| GOTERM_BP_FAT | GO: 0030855~epithelial cell differentiation | 48 | 2.328967 | 1.29E−15 |
| GOTERM_BP_FAT | GO: 0031424~keratinization | 26 | 1.261524 | 2.82E−15 |
| GOTERM_BP_FAT | GO: 0030216~keratinocyte differentiation | 32 | 1.552644 | 3.93E−15 |
| GOTERM_BP_FAT | GO: 0009913~epidermal cell differentiation | 33 | 1.601164 | 1.01E−14 |
| GOTERM_BP_FAT | GO: 0007267~cell-cell signaling | 116 | 5.628336 | 1.16E−13 |
| GOTERM_BP_FAT | GO: 0060429~epithelium development | 59 | 2.862688 | 1.50E−12 |
| GOTERM_BP_FAT | GO: 0007610~behavior | 92 | 4.463852 | 2.48E−11 |
| GOTERM_BP_FAT | GO: 0007155~cell adhesion | 121 | 5.870936 | 8.14E−11 |
| GOTERM_BP_FAT | GO: 0022610~biological adhesion | 121 | 5.870936 | 8.99E−11 |
| REACTOME_PATHWAY | REACT_13433: Biological oxidations | 27 | 1.310044 | 1.03E−05 |
| (Pathway) | | | | |
| REACTOME_PATHWAY | REACT_ 15518: Transmembrane transport of small molecules | 12 | 0.582242 | 0.001040232 |
| REACTOME_PATHWAY | REACT_14797: Signaling by GPCR | 74 | 3.59049 | 0.014661738 |
| REACTOME_PATHWAY | REACT_152: Cell Cycle, Mitotic | 40 | 1.940805 | 0.016734347 |
| REACTOME_PATHWAY | REACT_15314: Hormone biosynthesis | 11 | 0.533721 | 0.017783183 |
| SMART (Protein Domain) | SM00199: SCY | 20 | 0.970403 | 2.00E−09 |
| SMART | SM00020: Tryp_SPc | 29 | 1.407084 | 5.80E−06 |
| SMART | SM00389: HOX | 46 | 2.231926 | 9.54E−06 |
| SMART | SM00060: FN3 | 39 | 1.892285 | 1.69E−05 |
| SMART | SM00181: EGF | 40 | 1.940805 | 2.73E−05 |
| SMART | SM00120: HX | 11 | 0.533721 | 2.93E−05 |
| PFAM (Protein Domain) | PF00048: IL8 | 20 | 0.970403 | 6.14E−10 |
| PFAM | PF00048: interleukin-8 like | 17 | 0.824842 | 2.96E−08 |
| PFAM | PF00413: Peptidase_M10 | 17 | 0.824842 | 5.76E−07 |
| PFAM | PF00089: Trypsin | 29 | 1.407084 | 1.51E−06 |
| PFAM | PF00061: Lipocalin/cytosolic fatty-acid binding protein family | 13 | 0.630762 | 1.63E−06 |
| PFAM | PF01023: S_100 | 13 | 0.630762 | 1.63E−06 |
| PFAM | PF00061: Lipocalin | 14 | 0.679282 | 2.08E−06 |
| KEGG_PATHWAY | hsa04060: Cytokine-cytokine receptor interaction | 50 | 2.426007 | 1.15E−06 |
| (Pathway) | | | | |
| KEGG_PATHWAY | hsa04080: Neuroactive ligand-receptor interaction | 46 | 2.231926 | 1.75E−05 |
| KEGG_PATHWAY | hsa02010: ABC transporters | 14 | 0.679282 | 1.23E−04 |
| KEGG_PATHWAY | hsa00982: Drug metabolism | 17 | 0.824842 | 1.26E−04 |
| KEGG_PATHWAY | hsa00350: Tyrosine metabolism | 13 | 0.630762 | 5.10E−04 |
| KEGG_PATHWAY | hsa00140: Steroid hormone biosynthesis | 13 | 0.630762 | 7.91E−04 |
| KEGG_PATHWAY | hsa00980: Metabolism of xenobiotics by cytochrome P450 | 15 | 0.727802 | 9.98E−04 |

It is important to find enriched pathways in order to understand the underlying changes in cellular biology for specific samples. Enriched pathways were detected by pathway network analysis.

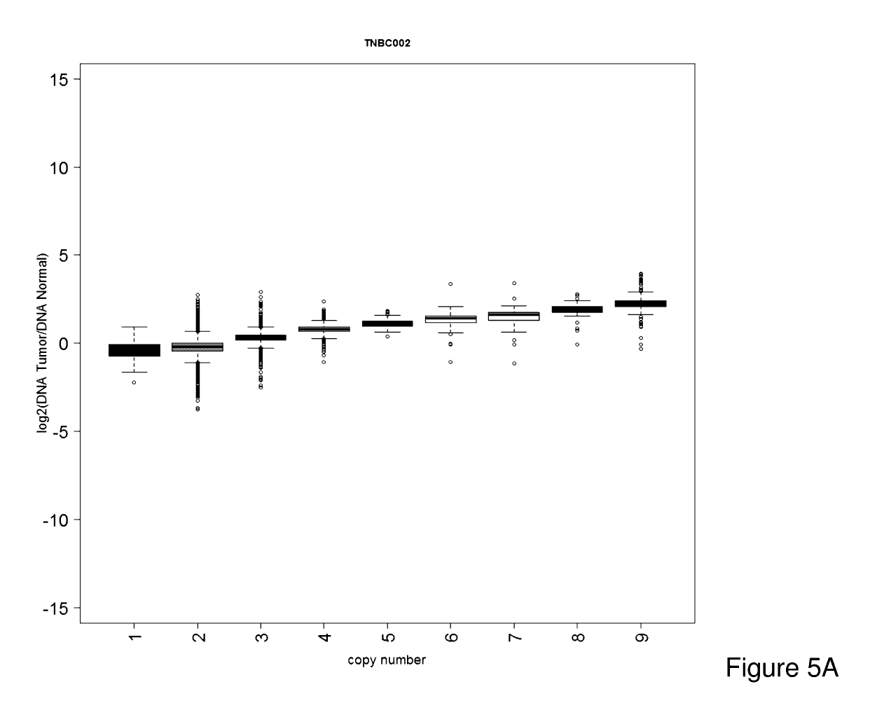

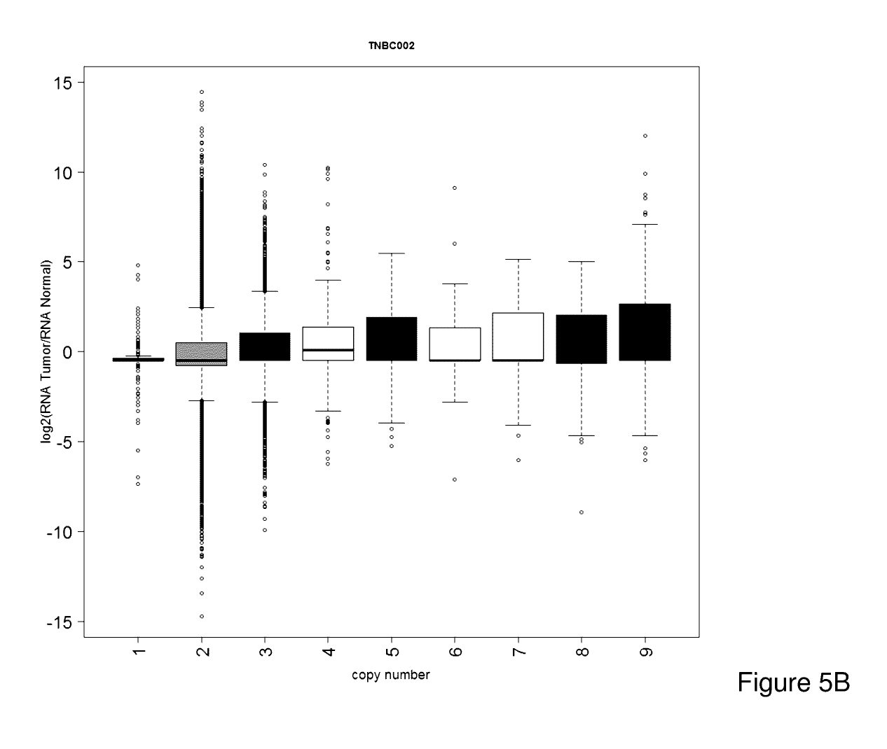

In FIG. 5, gene copy number (x-axis) versus gene (coding regions) coverage fold change between Tumor and Normal samples (y-axis) were compared in both the DNA libraries (FIG. 5A) and RNA libraries (FIG. 5B). In addition, copy number variation (genome level) was determined by comparing observed coverage in DNA libraries of tumor and (matched) normal samples. Reads aligning to coding regions (sources: Ensembl) from both DNA and RNA libraries were used for comparing coverage changes in the two types of libraries. An increased variation in (fold) changes of RNA libraries (transcription effect) was observed. DNA copy number and RNA expression have a significant correlation (F-statistic =80.88, p-value <2.2e-16). Correlation increases for higher copy number. 7.3% of RNA fold change variation can be explained by DNA copy number.

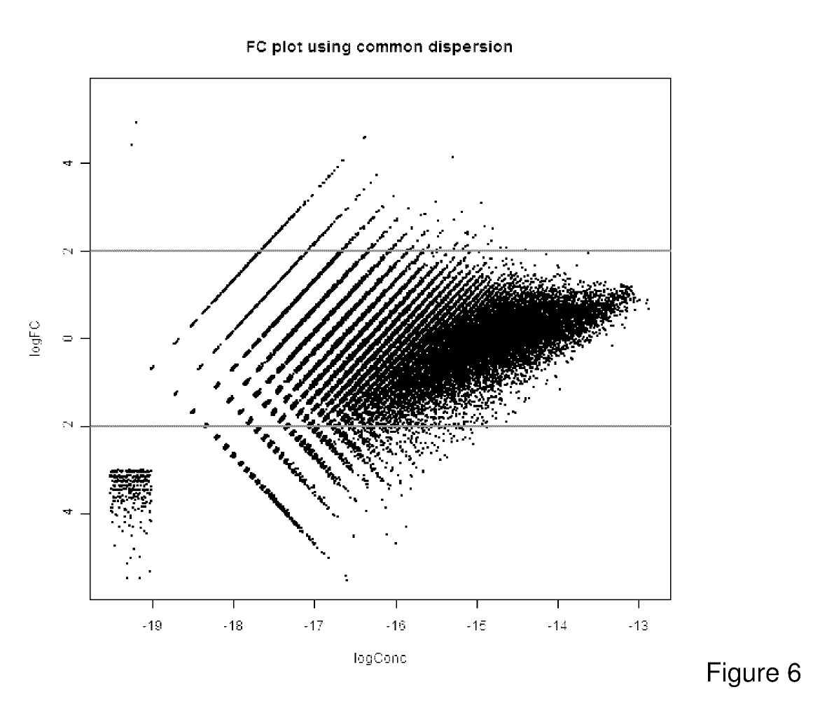

Splicing and gene fusion events were also called for using LifeScope 2.0™ WT package (Life Technologies, Applied Biosystems, Carlsbad, Calif.). Several known and putative splicing junctions were found that were up-regulated or down-regulated in multiple samples in comparison to RNA normals. The MA plot highlights the 500 most highly differentially expressed junctions (FIG. 6). A plot was generated using EdgeR (Robinson et al, 2010).

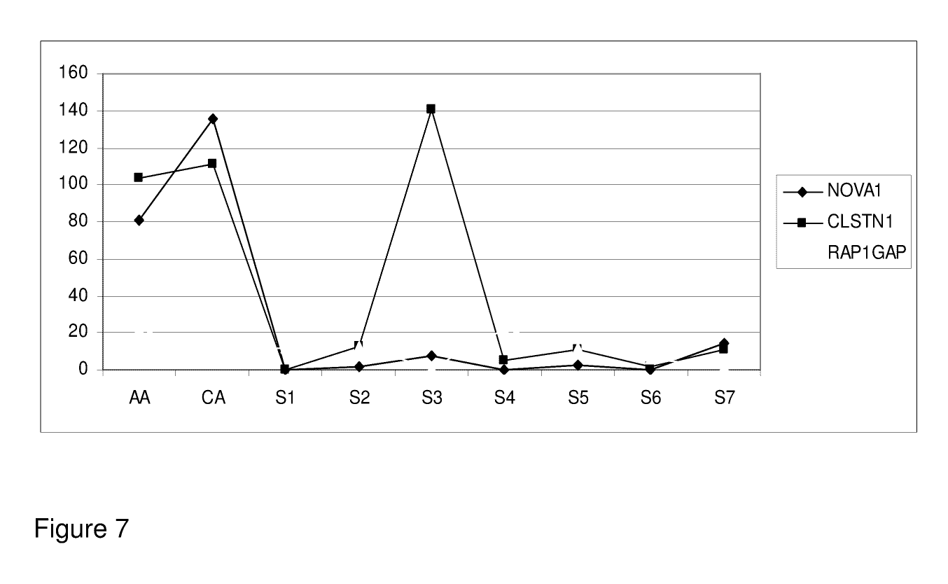

Among the alternative splicing regulations discovered above, transcription factors such as Noval silence or enhance exons on pre-mRNAs containing at least one type of YCAY cluster motif. An overall correlation between NOVA1 depletion (on 5 out of 7 TNBC samples) and its targets was observed. Two particular exons (CLATN1 and RAP1GAP) showing correlation of regulation with NOVA1 are shown in FIG. 7.

### Example 3

### Therapeutically Relevant Pathway and Therapeutic Targeting

The clinical application of next generation sequencing toT comprehensively characterize groups of driving mutations in individual metastatic triple negative breast cancer (mTNBC) genomes has the potential to reveal therapeutically relevant pathway dependencies (See Example 2 Table 6). Tissue from 14 patients with mTNBC were harvested and deep whole genome and transcriptome sequencing were conducted for each case to identify mutations that can guide therapeutic targeting within available phase I/II clinical trials.

As detailed in previous examples, the Life Technologies SOLiD system was utilized to sequence germline and tumor DNA to sufficient depth to identify somatic genome alterations including point mutations, indels, and structural events including translocations. Furthermore, RNA-seq was performed on these tumors, along with a series of age and ethnicity matched normal breast controls to perform deep differential expression analysis, isoform expression analysis, and fusion transcript detection. Investigational therapeutic options for each patient were examined by evaluating the sequencing findings.

The whole genome and transcriptome sequencing study revealed numerous known and novel mutations in mTNBC. All patients' cancers analyzed to date had alterations that would activate the MAPK pathway, however, through various mechanisms in different patients. These metastasized TNBC associated alterations include BRAF amplification and overexpression, NF1 homozygous deletion, and consistent IQGAP3 overexpression. Furthermore, all patients' cancers also harbor mutations that would activate the PI3K/AKT pathway. These mutations include PTEN homozygous deletion or down-regulation, consistent INPP4B down-regulation, FBXW7 homozygous deletion, and ERAS overexpression. Moreover, although it has been shown ERBB4 downregulation in breast tumors, an associated unique somatic genomic event that significantly alters the ERBB4 locus and leads to its loss on the RNA level in the majority (5/7) of our patients' tumors was provided herein (Table 10).

TABLE 10
Genomic Alterations and Pathways Enriched Thereof

| Gene | | UniProtKB/Swiss- | Somatic | Differential | Pathway |
|---|---|---|---|---|---|
| Name | Protein Name | Prot ID Number | Alteration | Transcription | involved |
| BRAF | Serine/threonine- | P15056 | amplification | overexpression | |
| | protein kinase | | | | |
| | B-raf | | | | |
| NF1 | Neurofibromin | P21359 | homozygous | | |
| | | | deletion | | |
| IQGAP3 | Ras GTPase- | Q86VI3 | | overexpression | |
| | activating-like | | | | |
| | protein IQGAP3 | | | | |
| PTEN | Mutated in | P60484 | homozygous | down- | activate |
| | multiple | | deletion | regulation | the |
| | advanced | | | | PI3K/AKT |
| | cancers 1 | | | | pathway |
| INPP4B | Type II inositol- | O15327 | | down- | activate |
| | 3,4-bisphosphate | | | regulation | the |
| | 4-phosphatase | | | | PI3K/AKT |
| | | | | | pathway |
| FBXW7 | F-box/WD | Q969H0 | homozygous | | activate |
| | repeat- | | deletion | | the |
| | containing | | | | PI3K/AKT |
| | protein 7 | | | | pathway |
| ERAS | Embryonic stem | Q7Z444 | | overexpression | activate |
| | cell-expressed | | | | the |
| | Ras | | | | PI3K/AKT |
| | | | | | pathway |
| ERBB4 | Receptor | Q15303 | intronic | down- | activate |
| | tyrosine-protein | | deletion of | regulation | the |
| | kinase erbB-4 | | approximately | | PI3K/AKT |
| | | | 4.3 kb | | pathway |

The isolation of these metastasized TNBC associated alterations make it possible to prioritize therapeutic targeting in chemotherapy-refractory mTNBC patients. Among the targeted therapies, it was observed that one patient with a high-level BRAF amplification/overexpression along with down-regulation of PTEN and INPP4B, had a major response to combined MEK plus AKT inhibitors on a phase I study (See Example 4).

Example 4--Triple Negative Breast Cancer personalized genomics trial

The primary objective of the clinical trial is to provide information that might suggest a therapeutic option for patients with metastatic (or locally recurrent) triple negative breast cancer (mTNBC), and to provide information relating to Time to Progress (TTP) for patients with molecular profiled prescription versus TTP on their prior therapy. The secondary objective is to conduct a comprehensive evaluation of genetic mutations in TNBC that may accelerate the development of rational and precise therapeutics; and to evaluate patient's best response to molecularly-selected therapy.

A patient eligible for clinical trial inclusion in this study had to meet all of the following criteria: (1) has a metastatic or locally recurrent triple negative breast cancer and is scheduled for medically indicated surgical biopsy or resection of disease; (2) have measurable or evaluable (nonmeasurable) disease based on results generated by RECIST v 1.1 (Eisenhauera, 2009) presented after surgical biopsy/resection. Following surgical resection, if the tumor sample is found to be inadequate for comprehensive molecular analysis, the patient would be deemed ineligible and would be replaced; (3) has received at least 1 prior chemotherapeutic regimen for their metastatic or locally recurrent TNBC prior to initiating the molecularly-selected therapy. There is no limit on the prior therapy for TNBC. Intervening therapies were strongly recommended to be held off if possible from the time of biopsy to the completion of sequencing so as not to change the cancer under the selective pressure of treatment, so that the sequencing results would be reflective of the current cancer; (4) is >18 years of age; (5) has an expected survival of at least 6 months, as estimated by the treating oncologist; (6) has planned surgical resection (indicated for the medical care of the patient) that will yield a minimum fresh/frozen tumor sample of 1 cm×1 cm×1 cm that will be available for molecular profiling analysis; (6) is agreeable to having a blood sample (10-20mL) drawn and analyzed to compare their normal genetic profile to that of their tumor sample; (7) has signed the most recent Patient Informed Consent Form and a Patient Authorization Form.

A patient would be excluded from this study if she meets any of the following criteria: (1) has breast cancer other than metastatic or locally recurrent TNBC; and surgical resection of the recurrent TNBC will render the patient as “no evidence of disease” (NED), and thus to be replaced; (2) has a history of heart disease, other conditions that would prevent treatment with a standard chemotherapeutic agent; (3) has evidence of CNS involvement that is progressing or that requires radiation, resection or steroid therapy; (4) has a serious uncontrolled intercurrent medical or psychiatric illness, including serious infection; (5) is pregnant or nursing; (6) is unable to comply with requirements of study.

Generally, 14 patients with previously treated triple negative breast cancer were included in the clinical study (Table 11). Samples were collected for sequencing with the SOLiD™ system (Applied Biosystems, Carlsbad, Calif.), and the patients were treated according to what is found after CLIA certification of the target.

TABLE 11
14 patients with previously treated triple negative breast cancer in the clinical study

| | Uniquely | | | | | |
|---|---|---|---|---|---|---|
| | Aligned | Uniquely | Unique | Number | | |
| Participant | Reads | Aligned | Genome | Germline | Percent | Ti/Tv |
| Sample | (billion) | Bases | Coverage* | Variants | dbSNP | Ratio** |
| TNBC-001 Normal | 2.05 | 93 Gb | 31X | 2.9 million | 80.8% | 2.08 |
| TNBC-001 Tumor | 1.96 | 89 Gb | 29X | — | — | — |
| TNBC-002 Normal | 2.01 | 89 Gb | 29X | 2.6 million | 90.2% | 2.02 |
| TNBC-002 Tumor | 2.12 | 97 Gb | 31X | — | | |
| TNBC-003 Normal | 2.25 | 101 Gb | 33X | 3.1 million | 80.8% | 2.05 |
| TNBC-003 Tumor | 2.03 | 92 Gb | 30X | — | — | — |
| TNBC-004 Normal | 1.66 | 74 Gb | 24X | 3.0 million | 80.9% | 2.05 |
| TNBC-004 Tumor | 1.84 | 83 Gb | 27X | — | — | — |
| TNBC-005 Normal | 1.98 | 90 Gb | 29X | 2.7 million | 90% | 2.02 |
| TNBC-005 Tumor | 1.97 | 89 Gb | 29X | | | |
| TNBC-006 Normal | 2.73 | 125 Gb | 40X | 3.2 million | 81.4% | 2.06 |
| TNBC-006 Tumor | 2.72 | 124 Gb | 40X | — | — | — |
| TNBC-007 Normal | 2.37 | 106 Gb | 35X | 3.4 million | 80.7% | 2.08 |
| TNBC-007 Tumor | 2.61 | 119 Gb | 40X | — | — | — |
| TNBC-008 Normal | 2.21 | 99 Gb | 32X | 2.7 million | 90% | 2.04 |
| TNBC-008 Tumor | 1.52 | 67 Gb | 22X | — | — | — |
| TNBC-009 Normal | 1.44 | 64 Gb | 21X | 2.4 million | 91% | 2.02 |
| TNBC-009 Tumor | 1.57 | 69 Gb | 22X | — | — | — |

Every tumor is genomically unique with SNVs, indels, translocations, inversions, amplifications, and deletions. For example, after all the analysis (detailed in previous examples), it was found that TNBC1 comprises 3.7 Million SNPs; 9,766 genomic SNVs; 41 SNVs that are coding missense or nonsense; Small insertions or deletions with 16 in the coding region and 2,727 in the genomic non-coding region; 20 tranaslocations with 4 within genes; and 2,867 large deletions or insertions with 16 crossing gene(s). As a further example, CTNNA1 (catenin cadherin associated alpha 1) homozygous deletions were observed in two African American patients.

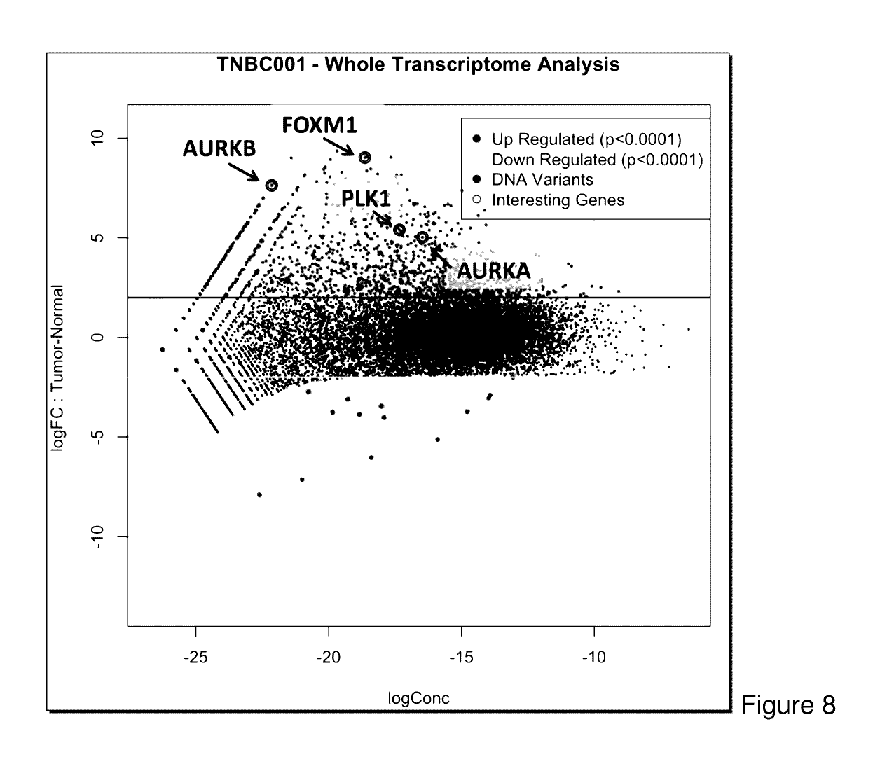

Further, TNBC001 whole transcriptome analysis identified significant up-regulation of AURKB, FOXM1, PLK1, AURKA (p<0.0001) (FIG. 8, also see Table 7). The TNBC-001 under-expressed genes identified through transcriptome RNA-seq are listed in Table 12.

TABLE 12
TNBC001 under-expressed genes

| | | UniProtKB/ | Log | log2 | |
|---|---|---|---|---|---|
| Gene | | Swiss-Prot | fold | (Tumor/ | |
| Name | Protein Name | ID Number | conc | Normal) | p. value |
| STAC2 | SH3 and cysteine- | Q6ZMT1 | −18.14 | −2.56 | 9.74E−07 |
| | rich domain- | | | | |
| | containing protein 2 | | | | |
| PTEN | Mutated in multiple | P60484 | −13.29 | −2.56 | 4.27E−07 |
| | advanced cancers 1 | | | | |
| EDA2R | Tumor necrosis | Q9HAV5 | −18.71 | −2.56 | 1.42E−06 |
| | factor receptor | | | | |
| | superfamily member | | | | |
| | 27 | | | | |
| DLL4 | Delta-like protein 4 | Q9NR61 | −17.79 | −2.56 | 8.32E−07 |
| TXK | Tyrosine-protein | P42681 | −21.38 | −2.57 | 8.17E−05 |
| | kinase TXK | | | | |

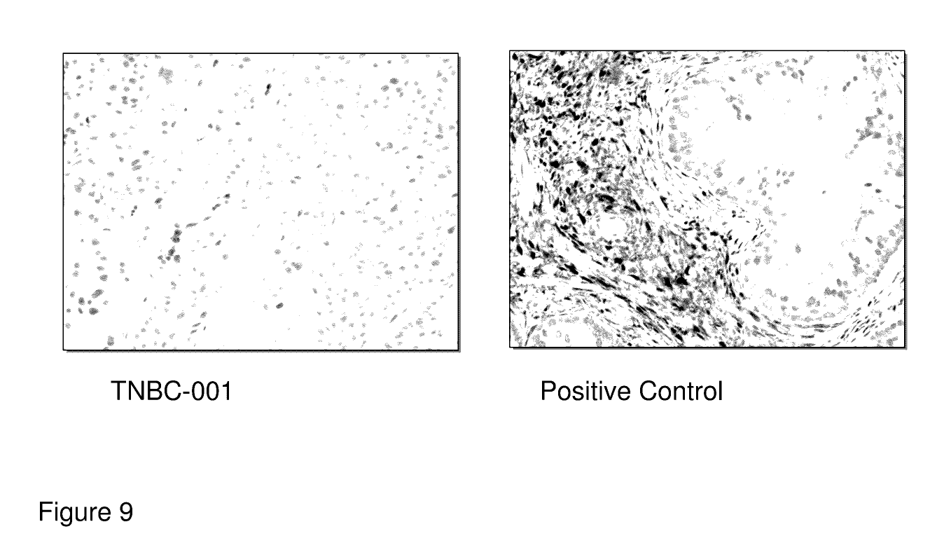

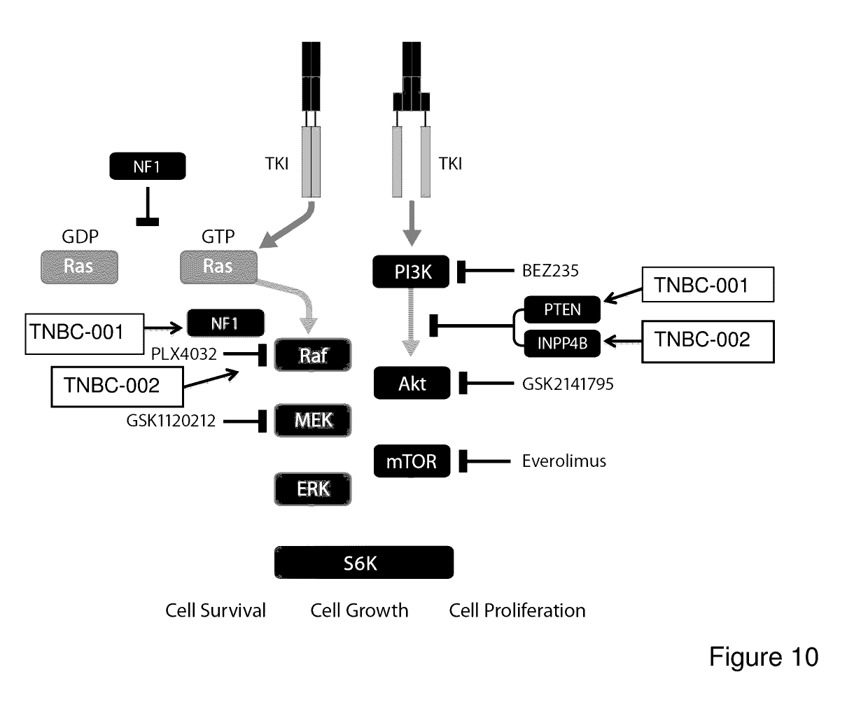

Among the listed genes in Table 13, TNBC-001 contains a PTEN Exon 6 homozygous deletion of about 1 kb. PTEN has clear evidence of expression in tumor, but with exon 6 deleted. However, PTEN has higher expression in normal breast, and all reads show normal splicing of exons 5, 6, and 7. Exon 6 deletion leads to transcript mutation of bases 493-634, resulting in frameshift mutation in premature truncation. In turn, the protein mutation is G165Ifs173X, standing for PTEN normal reading to amino acid 165, then frameshifts and truncates 8 bases later at amino acid 173. PTEN G165Ifs173X protein truncating mutation leads to complete protein loss (FIG. 9). In addition, TNBC-001 contains NF1 19 bp homozygous coding deletion. NF1, PTEN and INPP4B are all in the RAS/RAF/MEK/ERK and PI3K/AKT/mTOR pathway (FIG. 10).

In TNBC-002, high level BRAF amplification and increased mRNA expression were observed (Table 13). In addition, concomitant PTEN and INPP4B underexpression took place. BRAF was amplified by CGH (comparative genomic hybridization) and showed increased mRNA expression. INPP4A and PTEN were down in tumor at expression level, although there are no significant variants or alterations at CGH level. ERBB4 is down by expression in tumor and presents with a possibly causal nonsense SNV. Two genes that can make part of PI3K reg class IA show discordant expression changes. PIK3CD expression is up in tumor. There is no significant alteration in PIK3CA. FGFR1 is highly upregulated at expression level. FGFR3 does not show much of change. It is unknown what ligand is used by tumor from only tumor data. It also appeared that all EGF/ERBB receptors are down regulated. NRG3 is up at expression and genomic level, but its ligand expression level is down.

TABLE 13
TNBC-002 genomic alterations

| | | | | Differential | Pathway |
|---|---|---|---|---|---|
| | | UniProtKB/Swiss-Prot | Somatic | expression | with the |
| Gene Name | Protein Name | ID Number | Alteration | in tumor | lesions |
| BRAF | Serine/threonine-protein | P15056 | | Up | |
| | kinase B-raf | | | | |
| INPP4B | Type II inositol-3,4- | O15327 | no | Down | |
| | bisphosphate | | | | |
| | 4-phosphatase | | | | |
| PTEN | Mutated in multiple | P60484 | no | Down | |
| | advanced cancers 1 | | | | |
| ERBB4 | Receptor tyrosine-protein | Q15303 | nonsense | Down | |
| | kinase erbB-4 | | SNV | | |
| PIK3CD | Phosphatidylinositol- | O00329 | | Up | |
| | 4,5-bisphosphate | | | | |
| | 3-kinase catalytic | | | | |
| | subunit delta isoform | | | | |
| FGFR1 | Fibroblast growth | P11362 | | Up | |
| | factor receptor 1 | | | | |
| EGF/ERB | Epidermal growth | P00533 | | Down | |
| B receptors | factor receptor | | | | |
| NRG3 | Pro-neuregulin-3, | P56975 | amplification | Up | |
| | membrane-bound | | | | |
| | isoform | | | | |

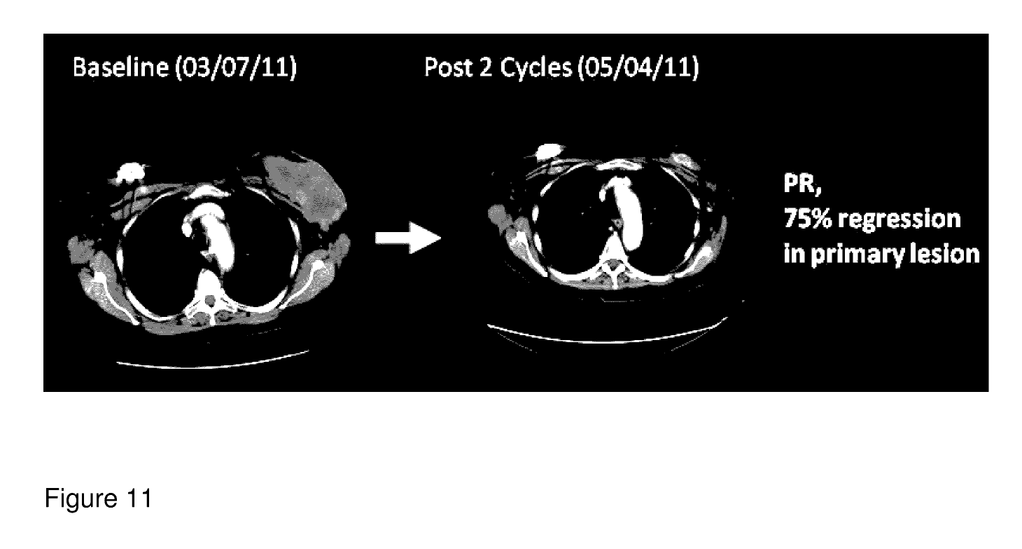

Patients, with completed with sequencing and with lesions found in both PI3K/AKT/mTOR and Ras/MEK/ERK pathways, were treated with dual pathway inhibitor combination: Glaxo Smith Klein agents trametinib and GSK2141795. The baseline was measured at the beginning of the treatment, after 2 cycles (close to two months), 75% regression in primary lesion were observed (FIG. 11).

In the case of TNBC-009 patient, multiple DNA damage and double-strand break repair defects were observed (Table 14). The patient was randomized on Iniparib (also called BSI 201) plus Gemcitabine and Carboplatin treatment and responded favorably.

TABLE 14
TNBC-009 DNA damage and double-strand break repair defects

| Gene | | UniProtKB/Swiss- | | |
|---|---|---|---|---|
| Name | Protein Name | Prot ID Number | Defect | Known Function |
| DCLRE1C | DNA cross-link | Q96SD1 | Mutation | Nuclear protein |
| | repair 1C protein | | (nonsynonymous) | involved in V(D)J |
| | | | | recombination |
| | | | | and DNA repair |
| MEI1 | Meiosis inhibitor | Q5TIA1 | Mutation | Double strand |
| | protein 1 | | (nonsynonymous) | break repair |
| TP53 | Cellular tumor | P04637 | Mutation | DNA Damage |
| | antigen p53 | | (nonsynonymous) | repair |
| DDB1 | DNA damage- | Q16531 | Mutation | Nucleotide |
| | binding protein 1 | | (nonsense) | Excision Repair |
| RIF1 | Telomere-associated | Q5UIP0 | Mutation | RIF1 contributes |
| | protein RIF1 | | (nonsynonymous) | to ATM-mediated |
| | | | | protection against |
| | | | | DNA damage. |
| ZBTB40 | Zinc finger and BTB | Q9NUA8 | Mutation | Phosphorylated |
| | domain-containing | | (nonsynonymous) | upon DNA |
| | protein 40 | | | damage, probably |
| | | | | by ATM or ATR. |
| BRIP1 | ATP-dependent RNA | Q9BX63 | Deletion | DNA double- |
| | helicase BRIP1 | | | strand breaks by |
| | | | | homologous |
| | | | | recombination in |
| | | | | a manner that |
| | | | | depends on its |
| | | | | association with |
| | | | | BRCA1 |

A total of 10 mTNBC tumors were completed with whole genome and transcriptome sequencing, and with clinical annotation and outcome. The analysis uncovered previously known and unknown DNA and RNA alterations associated with TNBC. Complicated solid tumors pathways rather than individual genes were shown herein to affect therapeutic strategy and outcome. Provided in the present study, the dual RAS/RAF/MEK/ERK & PI3K/AKT/mTOR activation by multiple mechanisms in different tumors may be common in mTNBC. Thus these two pathways are likely to be highly therapeutically relevant. In addition, multiple mutations in double strand break repair pathway in metastatic tumor was shown to be responsive to Iniparib.

In comparison, the Ion AmpliSeq Cancer Panel, designed by NCI, OHSU and some other leading cancer research institutions, contains 46 genes with 190 amplicons and 739 mutations, and over 100 of the mutations are in BRAF, EGFR and KRAS (Table 15).

Using the Ion AmpliSeg™ Cancer Panel, the expected cancer associated mutations in patients are entirely different (see Table 16). Further, no therapeutic suggestions or decisions were made from what is likely to be found on the current AmpliSeg™ Cancer Panel using these mutations or targets.

TABLE 16
The Ion AmpliSeq ™ Cancer Panel predicted associated genes
in patients based on the completed whole genome sequencing
in the study:

| Patient | AmpliSeq ™ Panel Expected Results |
|---|---|
| TNBC1 | RB1 (47 bp del) |
| TNBC2 | ERBB4(k1160I), TP53(K320X) |
| TNBC3 | NO MUTATIONS |
| TNBC4 | FGFR2(M186T), ATM(Q1689E) |
| TNBC5 | TP53(2bp del) |
| TNBC6 | FGFR2(P298T) |
| TNBC7 | TP53(E339X) |
| TNBC8 | FGFR1 (V247 L) |
| TNBC9 | PTPN11(Y197X), TP53(H193R) |
| TNBC13 | TP53(Y220C) |

In comparison, using the approach of pathway with enriched alterations, the treatments selected (Table 17) and the responses (Table 18) among the patients in the clinical trial are shown. Unique therapeutic treatments for an individual patient can be devised by detecting targetable pathways. As understood, using pathways enriched with gene alterations to make therapeutic decisions may mean frequent needs to get off-label use for drugs or obtain investigational agents.

TABLE 17
TNBC Biomarker Profile Guided Treatment Options

| | | | | Key Genes | | First | Second |
|---|---|---|---|---|---|---|---|
| ID | Ethnicity | Subtype | Key Events | in the Events | Actionable | Treatment | Treatment |
| 1 | African | BS1 | High proliferation | PTEN, RB1, NF1, | NF1 homozygous | BEZ235 pan | Doxil/Velcade |
| | America | expressing | cassette, ERBB4 | AURKA/B, PLK1, | insertion/deletion + | PI 3Ki adv | and Cytoxan |
| | | basal and | homozygous deletion, | FOXM1, TOP2A, | PTEN homozygous | solid-Novartis | |
| | | luminal | CTNNA1 homozygous | ERBB4 homozygous | insertion/deletion = | | |
| | | keratins | deletion, RB1, PTEN | deletion and | MEK/AKT | | |
| | | | homozygous | underexpression | pathway activation | | |
| | | | insertion/deletion, NF1 | | | | |
| | | | homozygous | | | | |
| | | | insertion/deletion | | | | |
| 2 | Caucasian | IM | High NFKB cassette, | BRAF amp hi, | BRAF | MEK/AKT | MEK/AKT |
| | American | expressing | TP53 mutated, ERBB4 | INPP4B lo, ERBB4 | amplification + | (GSK) | (GSK) |
| | | basal | mutated. Non-significant | mutated and lo | INPP4B under | | |
| | | keratins | BRCA2 mutation | | expression = | | |
| | | | | | MEK/AKT | | |
| | | | | | activation | | |
| 3 | African | BS1 | High proliferation | AURKA/B, PLK1, | TOP2A, PBK | Eribulin | Eribulin |
| | American | expressing | cassette, high amplified | FOXM1, TOP2A, | overexpression | | |
| | | basal | WT1/WIT1, PBK over | PBK hi, ERBB4 | | | |
| | | keratins | expression, TP53 | homozygous deletion | | | |
| | | | mutated, ERBB4 | and under expression | | | |
| | | | homozygous | | | | |
| | | | insertion/deletion | | | | |
| 4 | African | BS1 | High proliferation | Hi IQGAP3, | IQGAP3 | Doxil/Velcade | BEZ235 pan PI |
| | American | expressing | cassette, high WT1 | AURKA/B, PLK1, | overexpression + | and Cytoxan | 3Ki adv solid- |
| | | basal | | FOXM1, TOP2A, | INPP5F inversion + | | Novartis |
| | | keratins | | INPP5F Complex | NEDD4 mutation = | | |
| | | | | inversion and point | MEK/AKT | | |
| | | | | mutation, NEDD4 | activation | | |
| | | | | mutation, ERBB4 | | | |
| | | | | under expression | | | |
| 5 | Caucasian | M | EMT with ALK and | WT1 overexpression, | IQGAP3 | BEZ235 | BEZ235 |
| | American | expressing | Notch hi, DKK1 hi (wnt), | ALK overexpression, | overexpression + | | |
| | | basal | WT1 hi, IQGAP3 hi, | IQGAP3 | INPP4B | | |
| | | keratins | CCNE1hi, ERBB4 lo, | overexpression, | underexpression, | | |
| | | and high | hi/medium proliferation | CCNE1 | AKT3 | | |
| | | proliferation | | overexpression, | overexpression = | | |
| | | genes | | INPP4B | MEK/AKT; ALK | | |
| | | | | underexpression, | overexpression = | | |
| | | | | AKT3 overexpression | ALK inhibitor; | | |
| | | | | | CCNE1 | | |
| | | | | | overexpression = | | |
| | | | | | curcumin | | |
| 6 | African | BS1 | High proliferation | AURKA/B, PLK1, | GART | MEK/AKT | Pemitrexed |
| | American | expressing | cassette, ERBB4 homz | FOXM1, TOP2A, | overexpression = | (GSK) | (Alimpta) |
| | | basal | del, IQGAP3 hi, PBK hi, | GART, IQGAP3 | Pemitrexed | | |
| | | keratins | TP53 mutated, HDAC6 | overexpression, PBK | (Alimpta) | | |
| | | | mutated | overexpression | | | |
| 7 | African | BS1 | High proliferation | FBXW7 + INPP4B lo, | FBXW7 + INPP4B = | Currently on: | Currently on: |
| | American | expressing | cassette, CTNNA1 homz | TOP2A mutated, | AKT/MTOR | T0925 | T0925 |
| | | basal | del, FBXW7 homz del, | AURKA/B, PLK1, | activation; TOP2A | Taxol + Avastin + | Taxol + Avastin + |
| | | keratins | WHSC1L1 amp and hi, | FOXM1 | mutated and | Everolimus/ | Everolimus/ |
| | | with | PBK hi (WHSC1L1 | | overexpression = | placebo (pill) | placebo (pill) |
| | | family | possible germline | | etoposide (sens or | | |
| | | history | abnormality) | | resist?) | | |
| 8 | Caucasian | BS2 or | DNA repair mutations in | DNA repair mutations in | DNA repair | | Gem/Carpbo/ |
| | American | Unc | TOP2A, RAD23A, | TOP2A, RAD23A, | mutations = PARP | | Iniparib |
| | | expressing | MDM2,BRD4, UBR2 | MDM2, BRD4, | inhibitor | | |
| | | basal | mutated, VEGFA hi, | UBR2 mutated, | | | |
| | | keratins | MET hi, NMYC hi, | VEGFA/MET, | | | |
| | | with MET | HSP90AB1 hi, KRAS hi | NMYC, | | | |
| | | and Myc | | HSP90AB1,KRAS | | | |
| | | expression | | | | | |
| 9 | Caucasian | BS1 with | DNA repair mutations | DNA repair mutations | DNA repair | AC/T/bilat | Gem/Carpbo/ |
| | American | family | ZBTZ40, DCLRE1C, | ZBTZ40, DCLRE1C, | mutations = PARP | mastec close to | Iniparib |
| | | history | DDB1, LTBR, TP53, | DDB1, LTBR, TP53, | inhibitor | path complete | |
| | | | RIF1, MEI1 | RIF1, MEI1 | | response | |

Comprehensive genomic and transcriptomic interrogation of mTNBCs revealed events supporting co-activation of the MAPK and PI3K/AKT pathways in all the tumors, albeit by different mutational mechanisms and supports potential effectiveness of combination therapy in the treatment of mTNBC. These patients may be treated with combined mek plus akt inhibitors to determine the effectiveness of co-inhibition of these pathways based on this frequent genomic context.

TABLE 18
TNBC Biomarker Profile Guided Treatment Responses

| Patient # | Treatments | Best results |
|---|---|---|
| 1 | BEX235 (hit only one arm of what | Stable Disease |
| | suggested MEK/AKT | |
| 2 | GSK MEK/AKT inhibitor | Partial Response |
| 3 | BEZ 235 | Progressive Disease |
| 4 | Eribulin | Stable Disease |
| 5 | ENZ 2208 (TOPO1) | Partial Response |
| 6 | Taxol + Avastin + Everolimus | Complete Response |
| 7 | GSK MEK/AKT | Progressive Disease |
| 8 | BSI 201 + gemcitabine + carboplatin | Partial Response |
| 9 | BSI 201 + gemcitabine + carboplatin | Complete Response |

### Example 5

### Therapeutically Actionable Molecular Concepts in mTNBC Using Integrated analysis of mTNBC genome and transcriptome data

### Description of Samples and NGS Data Quality Assessment

Fresh frozen relapse refractory tumor specimens from clinical biopsy and peripheral blood from 14 women clinically diagnosed with mTNBC were collected. Seven of the patients are African American (AA), and seven are European American (EA). Demographics and clinical treatment history for these patients is provided in Table 19.

TABLE 19
Study participant statistics

| Participant | Ethnicity¹ | Comments on Clinical Treatment History |
|---|---|---|
| ID | Age² | |
| mTNBC1 | AA 53 | CR: preop AC |
| | | PR: iniparib/gemcitibine/carboplatin |
| | | PD: bortezomib/cyclophosphamide/pegylated |
| | | liposomal doxorubicin |
| mTNBC | EA 60 | PR: iniparib/gemcitabine/carboplatin |
| mTNBC3 | AA 47 | SD: iniparib/gemcitabine/carboplatin |
| mTNBC4 | AA 58 | PD: iniparib/gemcitabine/carboplatin |
| | | PD: bortezomib/cyclophosphamide/pegylated |
| | | liposomal doxorubicin |
| mTNBC5 | EA 58 | PR: preop AC then PD: preop docetaxel |
| | | PD: iniparib/gemcitabine/carboplatin |
| mTNBC6 | AA 32 | No response to preop A/CT |
| mTNBC7 | AA 44 | Primary refractory to all cytotoxic agents |
| | | CR: paclitaxel/bevacizumab for 8 mos |
| mTNBC8 | EA 63 | Bone-only metastatic disease at presentation |
| mTNBC9 | EA 40 | Clinical CR: preop AC/T |
| mTNBC10 | AA 59 | PR: preop AC/T |
| | | PD: iniparib/gemcitabine/carboplatin |
| mTNBC11 | EA 35 | Primary refractory to neo/adjuvant AC/T |
| | | PD: iniparib/gemcitabine/carboplatin |
| | | Rapid death marrow & CNS mets |
| mTNBC12 | EA 64 | Bone & diffuse adenopathy at presentation |
| mTNBC13 | EA 64 | BRCA2 mutation; h/o ovarian ca |
| | | (paclitaxel/carboplatin) and bilateral breast |
| | | cancer |
| | | PD: ixabepilone/capecitabine |
| mTNBC14 | EA 55 | Regional lymphadenopathy primary refractory |
| | | to all cytotoxic agents and radiation |

¹AA = African American, EA = European American,
²Age at diagnosis;
CR: Complete Response;
PR: Partial Response;
SD: Stable Disease;
PD: Progressive Disease.

SOLiD version 4 (50 bp) mate pair reads with 1.5 kb inserts were utilized for sequencing of germline and tumor DNA. Genome sequence coverage typically was above 30× for both tumor and germline genomes. Whole transcriptome sequencing (RNA-seq) was performed on high quality total RNA extracted from each tumor specimen, and from fresh hyperplastic breast specimens. On average 43 million (83%) RNA reads uniquely mapping to the genome/transcriptome were generated.

Somatic variants were detected using a pipeline consisting of multiple tools and described in detail in Supplementary Methods. Somatic mutations analyzed included single nucleotide variants (SNVs), indels, translocations, intrachromosomal rearrangements (inversions, etc), and copy number alterations. Two independent differential gene expression analyses were performed. One analysis was performed to determine differential expression between mTNBC tumors and nonmalignant hyperplastic breast samples using EdgeR (Robinson et al, 2010). A second analysis was based on a ‘leave one out’ method to assess differential expression for each tumor when compared against other tumors within our cohort. Integrated genome/transcriptome analyses were performed to assess allele specific expression between tumor DNA and tumor RNA using Bayesian analysis for shifts in the proportion of mutant alleles and reference alleles within RNA given an event at the DNA level.

Finally, genomic results, knowledge mining, pathway tools, and drug space were leveraged to link therapeutically actionable events or concepts with known drugs available through clinical trials to inform therapy for chemoresistant tumors.

Integrated Analysis of mTNBC Genome and Transcriptome Data

To identify somatic events in each tumor, germline/tumor paired genome analyses were performed to uncover point mutations and structural events within tumor genomes. As shown in Table 20 by the number of events, coding mutations were identified within the sample set, which include point mutations and small indels.

TABLE 20
Genetic variant summaries for each patient.

| | | | | | | | | Point Indels | | | Focal ³ | | | |
|---|---|---|---|---|---|---|---|---|---|---|---|---|---|---|
| | Germline SNPs | | | Somatic SNVs | | | | | Intronic | | Del | Amp | Trans. | |
| Par | Total % dbSNP Ti/Tv | Total | ssjSNPs | nsSNP | ssSNP | Intronic | UTR | Total | Coding | UTR | Total | Total | Total | Genic⁴ |
| 1 | 2.9M/81%/2.1 | | 6 | 30 | 8 | 1773 | 48 | 1298 | 5 | | 19 | 30 | 22 | 16 |
| 2 | 2.6M/90%/2.1 | | 6 | 10 | 11 | 2913 | 75 | | 2 | | 8 | 127 | 28 | 20 |
| 3 | 3.1M/80%/2.1 | | 2 | 46 | 12 | 1440 | 44 | | 0 | | 7 | 20 | 3 | 1 |
| 4 | 3.0M/81%/2.1 | | 11 | 52 | 22 | 4515 | 91 | | 3 | | 1 | 10 | 5 | 4 |
| 5 | 2.7M/90%/2.0 | | 15 | 47 | 28 | 4887 | 132 | | 3 | | 0 | 17 | 7 | 2 |
| 6 | 3.2M/81%/2.1 | | 31 | 110 | 33 | 6745 | 169 | | 6 | | 14 | 23 | 4 | 4 |
| 7 | 3.4M/81%/2.1 | | 20 | 35 | 49 | 7013 | 188 | | 5 | | 1 | 16 | 8 | 3 |
| 8 | 2.7M/90%/2.0 | | 9 | 31 | 15 | 4900 | 134 | | 3 | | 0 | 11 | 2 | 2 |
| 9 | 2.4M/91%/2.0 | | 7 | 7 | 28 | 3801 | 92 | | 4 | | 6 | 122 | 7 | 5 |
| 10 | 1.9M/82%/2.0 | | 1 | 58 | 10 | 1699 | 36 | | 16 | | 0 | 1 | 6 | 5 |
| 11 | 2.6M/90%/2.1 | | 7 | 21 | 22 | 4364 | 72 | | 6 | | 13 | 61 | 24 | 19 |
| 12 | 2.8M/90%/2.1 | | 3 | 23 | 17 | 2890 | 54 | | 3 | | 0 | 24 | 41 | 24 |
| 13 | 2.4M/91%/2.0 | | 1 | 29 | 13 | 2080 | 57 | | 3 | | 19 | 25 | 3 | 0 |
| 14 | 2.3M/91%/2.0 | | 8 | 18 | 26 | 2609 | 73 | | 6 | | 10 | 20 | 8 | 4 |

¹ dbSNP129;
² Ti/Tv = Transition/Transversion rate;
³ Focal events are amplifications and deletions that are less than 15 Mb and greater than 1.5 Kb, with a log2 fold-change greater than 0.5.
⁴Genic refers to when one of two breakpoints occurs between promotor and 3′ UTR.

On average 9.3 mutations per megabase in the cohort were detected. Importantly, there were no recurring point mutations that occurred at the same nucleotide residue in multiple tumors. Leveraging RNA-seq data, the expression of somatic SNVs at the transcript level using RNA-seq data were also assessed, by calculating the number of mutations that were present in both DNA and RNA versus those only present in DNA. In this analysis all data points were taken including situations where a gene harbored a somatic SNV, but the gene was essentially not expressed at all at the RNA level. The mean percentage of somatic SNVs that were expressed across our 10 samples was 34% (range 22%-60%).

As has been previously described in basal-like breast tumors, TP53 mutations were most frequent in this mTNBC dataset, observed in 9 of 14 mTNBC tumors. This included four tumors with nonsysnonymous substitutions, three with nonsense mutations, one with an indel, and one tumor with a mutation in a conserved splice consensus sequence. In all cases of TP53 mutation, loss of heterozygosity (LOH) was detected without hemizygous deletion of 17p or the TP53 locus, suggesting uniparental disomy as the likely mechanism for TP53 LOH in mTNBC. Extending to a genome-wide analysis of allele specific expression between the tumor RNA and tumor DNA, TP53 was the only gene with more than two somatic mutations exhibiting significant transcriptional allelic imbalance. Integrating RNA-seq data revealed that the frequency of the mutated allele exhibited transcriptional allelic inbalance, where the mutated allele was expressed above 90% in all but two tumors. Further, this was observed in the face of upwards of >30% normal contaminating stroma in samples used for analyte extraction. This observation is consistent with the absence of expression of wildtype TP53 within neighboring stromal contaminating tissue as has been previously reported, for which the actual mechanism remains unknown. Recent studies on brain tumors show similar monoallelic expression of TP53 due to LOH have prognostic value towards outcome.

Fifteen additional genes harbored somatic nonsynonymous or consensus splice site SNVs and/or small indels in more than one tumor. Among these genes included HERC1 (n=2), LRP1B (n=2), TOP2A (n=2), and CDH5 (n=3). HERC1 acts as an E3 ubiquitin ligase (Q15751), and is known to interact with and destabilize the tumor suppressor TSC2. Mutations within the LRP1B gene (Low-density lipoprotein receptor-related protein 1B, Q9NZR2) were also detected in multiple tumors. LRP1B mutations have been reported to be frequent in non-small cell lung carcinoma. TOP2A (DNA topoisomerase 2-alpha, P11388) controls the topological states of DNA and is altered in cancer. Importantly, mutations in TOP2A have been associated with drug resistance. CDH5 (Cadherin-5, P33151) is a member of the cadherin family of cell adhesion molecules, and this cadherin is believed to play a critical role in endothelial cell biology.

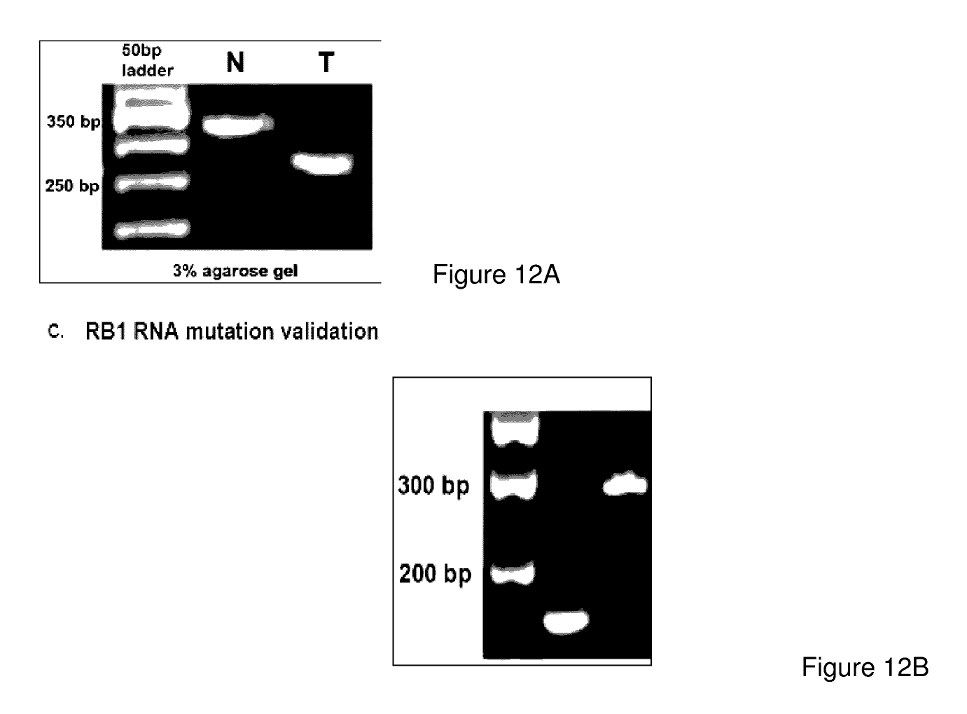

Aside from point mutations, additional small to moderately sized coding deletions (2 bp-5 kb) were also detected. Among genes containing somatic deletions in more than one tumor were RB1 and PTEN, both of which are known tumor suppressor genes (Table 18). Importantly, combined genome and transcriptome data allows for immediate interpretation of the consequence of deletions. One example was an RB1 49 bp deletion in mTNBC1 that includes 47 bases of exon 12, and extends to also delete the two nucleotides (GT) that define the conserved splice acceptor site (FIG. 12A). Therefore, this mutation could possibly result in either a frameshift, or a splicing error. PCR and Sanger sequencing were performed to validate the somatic DNA mutation and the resulting mutated cDNA confirming an in-frame splicing event of RB1 exon 11 to exon 13 (FIG. 12B). Importantly, this exon skipping results in an in-frame RB1 transcript, but with 44 amino acids deleted within the conserved RB domain. Importantly, this deleted RB1 transcript is expressed at normal levels by differential expression analysis methods. Thus, the use of a 3′ probe microarray would suggest normal RB1 expression, however this form of RB1 is likely non-functional due the loss of a large number of amino acids within a highly conserved domain. Conversely, a large ˜1 kb homozygous deletion encompassing exon 6 of PTEN also contained the consensus splice sites. However, in this case, exon skipping let to a frameshift and premature truncated form of PTEN, leading to complete loss of PTEN expression validated by RNA-seq expression and immunohistochemistry.

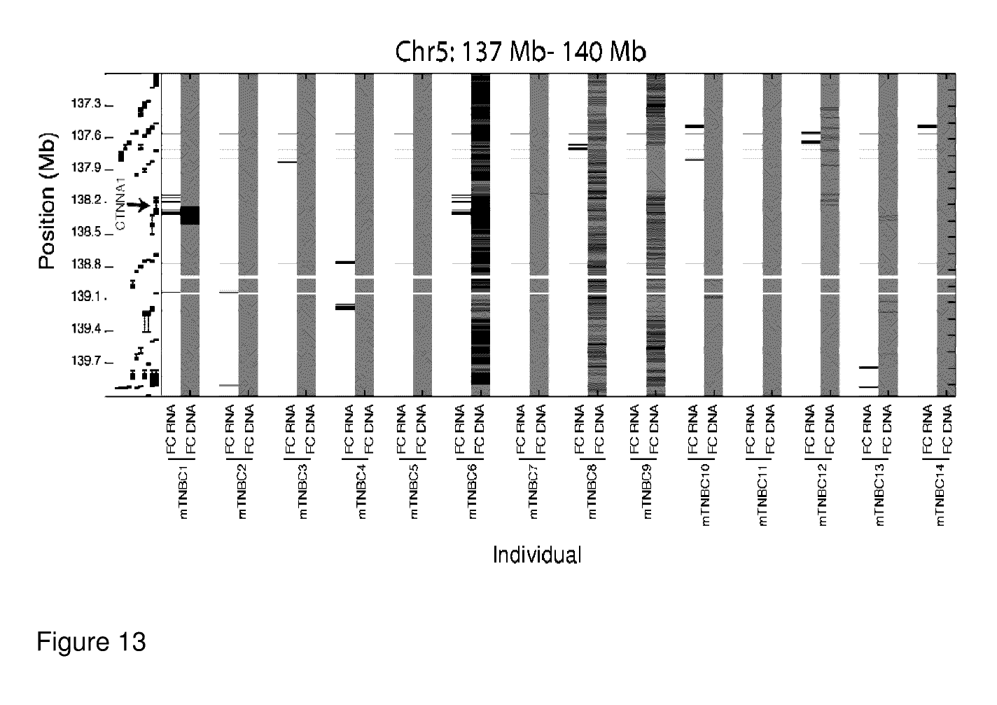

To assess large copy number changes across the genomes of mTNBC tumors, an algorithm that calculates the log2 ratio was utilized for the normalized read coverage from the germline genome data and normalized read coverage from the tumor genome data based on a 500 bp window. The most common region of broad copy number loss was at 5q, while copy number gains were observed most commonly at 1q, 6p, 8q, and 10p. Focal events were emphasized, described herein as log2FC copy number gains of ≧1.5 from neutral and less than 15 Mb in length, or homozygous deletions les than 1 5Mb in length. Among the focal copy number events that occurred in more than one tumor were unique homozygous deletions in two tumors (mTNBC1, mTNBC6) that involved the adjacent CTNNA1 and SIL1 loci at 5q31.2 (FIG. 13).

Integration of RNA-seq differential expression analyses using both comparison against nonmalignant samples and using our outlier analysis showed significant down-regulation of CTNNA1 (Catenin alpha-1; P35221) but not SIL1 (Nucleotide exchange factor SILL Q9H173) in both tumors harboring homozygous deletions. Therefore, these data strongly suggest CTNNA1 as the target tumor suppressor in mTNBC. The two tumors in this study that exhibit CTNNA1 loss were both from African American patients. In a previous study describing somatic alterations by whole genome sequencing of both a primary and metastatic lesion from a single 44 year-old African American patient with refractory TNBC, a homozygous deletion was also detected at the CTNNA1/SIL1 locus. This raises the question as to whether or not homozygous deletions of CTNNA1 are enriched in TNBC tumors with metastatic potential, particularly among African American patients. CTNNA1 forms a complex to anchor E-cadherin to the cytoplasmic cell membrane to maintain normal cell adhesion properties. Aberrations deregulating this complex result in dissociation of cancer cells from tumor foci, and represent a key primer for invasion and metastasis. Furthermore, a neuronal knockout model of alpha E-catenin demonstrated abnormal CNS cell growth with spreading of ventricular zone cells throughout the brain, which formed invasive tumor-like masses similar to those seen in human CNS tumors such as medulloblastoma, neuroblastoma, and retinoblastoma. Therefore taken together, these data suggest an important role for CTNNA1 loss in TNBC with invasive metastatic behavior, and begs the question concerning enrichment of this aberration among African American patients.

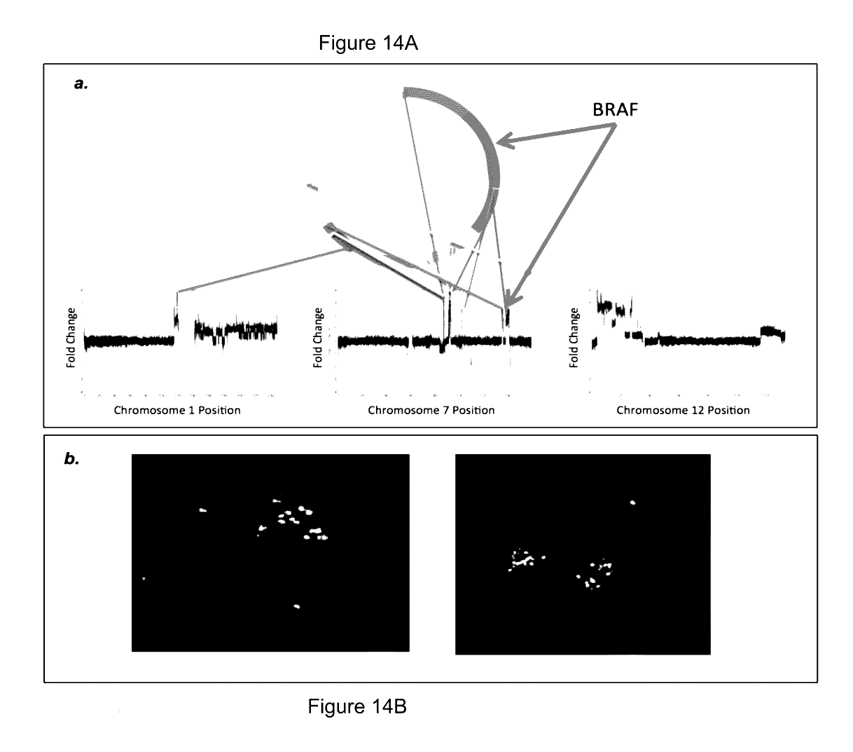

Several regions of amplification were identified within this cohort of mTNBC that encompass important oncogenes including WT1/WIT1, IQGAP3, IRS2, MYC, WHSC1L1/FGFR1, MYB, ARAF, and BRAF. A XMb region detected as a copy number amplification at 7q34 in the tumor from mTNBC2 contained the BRAF locus. An increased BRAF expression was also detected from RNA-seq data when comparing against nonmalignant controls (logFC=1.7) or when comparing across tumors using the outlier analysis (logFC=2.2). Upon further inspection of mate-pair genome sequence reads spanning the chromosome 7 breakpoints, it was evident that this putative amplicon was actually part of a more complex rearrangement illustrated in FIG. 14. Deconvolution of these data suggest that this rearrangement was likely a circular extrachromosomal double minute that includes chromosome 7 regions including the BRAF oncogene along with genomic material from chromosomes 1 and 12 (FIG. 14A). To validate the presence of the putative BRAF-containing double minute, interphase fluorescent in situ hybridization (FISH) on paraffin sections from this and other tumors using a BAC clone containing the BRAF locus were performed and the results confirmed the presence of amplified double minutes containing BRAF (FIG. 14B). To our knowledge this is the first report of BRAF amplification and double minutes in a TNBC tumor. And regardless of frequency, these findings have strong therapeutic implications.

Our approach has also uncovered additional cis and trans intrachromosomal rearrangements (inversions) and translocations. Notable alterations included two tandem overlapping inversion events within the INPP5F locus in mTNBC3. As both inversions span multiple exons of the INPP5F locus, it is likely that this structural alteration would lead to significant gene disruption in this tumor. Furthermore, copy number analysis shows copy number neutrality, therefore CGH analysis would not detect this rearrangement. INPP5F (Q9Y2H2) encodes inositol polyphosphate 5-phosphatase F, which modulates AKT/GSK3B signaling by decreasing AKT and GSK3B phosphorylation. Therefore, loss of INPP5F through structural events may lead to aberrant AKT signaling. These types of events generally go undetected in genomic studies using array-based or microscopy-based technologies.

A series of distinct somatic alterations at the ERBB4 locus in 3/14 tumors are of importance. These include a 4 kb intronic homozygous deletion (mTNBC1), a somatic point mutation (mTNBC2), and a breakpoint defining a larger 21Mb rearrangement at 2q34-q37.1 (mTNBC6). Furthermore, significant down-regulation of ERBB4 in all mTNBC in our study were observed, when comparing mTNBA tumors against nonmalignant controls. These data show that ERBB4 is frequently altered in mTNBC. The role of ERBB4 (Receptor tyrosine-protein kinase erbB-4, Q15303) in mammary physiology, including maturation of mammary glands during pregnancy and lactation through StatS activation is well known. ERBB4 mutations have been reported in multiple tumor types including lung carcinoma and melanoma, where mutations are believed to be oncogenic. However, ERBB4 also has been implicated as a tumor suppressor with growth inhibitory functions, and reactivation of epigenetically silenced ERBB4 using 5-aza-2′-deoxycytidine resulted in increased apoptosis in BT20 breast cancer cells. However, to our knowledge, this is the first report of somatic alterations at the ERBB4 locus in TNBC.

Therapeutically Actionable Molecular Concepts in mTNBC

Through the deep molecular profiling study described herein, a series of somatic events within individual tumors were detected that have informed or may have predicted the effectiveness of targeted therapies for some patients. Table 21 provides key information on therapeutically actionable concepts and clinical outcomes in the patient cohort used for this example.

TABLE 21
Key genomic alterations, therapuetic interventsion, and outcomes in mTNBC
patients

| | Amp¹ (↑) | | Struct. | | | Best response |
|---|---|---|---|---|---|---|
| Patient | Del² (↓) | Mutated | Variant | Differentially Expressed | Treatment | Disease Site |
| 1 | CTNNA1↓, PTEN↓, | NF1 | | EGF↑ HSP90AA1↑ RASGRF1↑ | BEZ235 | SD |
| | | RB1 | | TFDP1↑ MDM4↑ CCNE2↑ AR)↑ | (PI3K/mTOR | Chestwall |
| | | | | CTNNA1↓, NF1↓ | inhibitor) | |
| 2 | BRAF↑, MYC↑ | TP53 | | (BRAF, NRG3, NFKB2, PARP1, | GSK | PR |
| | | ERBB4 | | NFKBIA, HRAS)↑ | MEK/AKT | breast |
| | | | | | inhibitors | |
| 3 | SMAD3↑ SMAD6↑ | INPP5F | INPP5F | (PDGFRB, VEGFC, PDGFRA, | BEZ235 | PD |
| | | | | GLI2, VEGFA)↑ | (PI3K/mTOR | lung |
| | | | | | inhibitor) | |
| 4 | WT1↑, KLF8↑ | TP53 | | (WT1, PAK7)↑ | Eribulin | SD |
| | | | | (ZBTB16/PLZF)↓ | | lung |
| 5 | IQGAP3↑ | LRP1B | | (IL2, ALK, AKT2, CBLC, CCNE1, | EZN 2208 | PR |
| | | | | MYCN)↑ | (TOPO1 | nodes |
| | | | | (SFN[14-3-3sigma])↓, | inhibitor) | |
| 6 | WHSC1L1↑ FGFR1↑ | TOP2A | ERBB4 | (ID2, RPRM, EREG)↑ | Paclitaxel + | CR |
| | FBXW7↓ CTNNA1↓ | FGFR2 | | (FBXW7, CTNNA1, DKK1)↓ | bevacizumab + | chest wall |
| | | | | | everolimus | |
| 7 | NOTCH2↑ | TP53 | | (NOTCH2, KIT, GHR, TGFB2, | GSK | PD |
| | | DNER | | MMP9)↑ | MEK/AKT | chest wall |
| | | | | (CDKN2A, SFN[14-3-3sigma])↓ | inhibitors | |
| 8 | CAMK2D↑ | TP53 | | CAMK2D, ATG5, PTGS2, MET, | Iniparib + | Response |
| | SP90AB↑ | MDM2 | | TGFB2, AR, PDGFRA)↑ | gemcitabine + | bone |
| | ATG5↑ | TOP2A | | | carboplatin | |
| | | BRD4 | | | | |
| | | CDH5 | | | | |
| 9 | BRCA2↓ | TP53 | RB1 | (ERCC4, CCNE2, CDK1, MET, | Iniparib + | CR |
| | | DCLRE1C | PTEN | SMC1B, ALK, MYC)↑ | gemcitabine + | nodes |
| | | DDB1 | BRIP1 | (BRCA1, BRCA2)↓ | carboplatin | |
| | | RIF1 | | | | |
| | | MEI1 | | | | |
| 10 | KRAS↑ | TP53 | | (KRAS, DNTT)↑ | EZN 2208 | PR |
| | | CDH5 | | (ID4, SFRP1)↓ | (TOPO1 | chest wall |
| | | | | | inhibitor) | |
| 11 | PTEN↓ | BRCA1\hat{ }, | RB1 | (NOTCH3, TERT, CCNE2, | Iniparib + | PD |
| | | PTCH2 | RHEB | NFKB1, MET, RET)↑ | gemcitabine + | marrow/C |
| | | | | (NFATC2, TRAF3, SFRP1, DTX1, | carboplatin | NS |
| | | | | PTEN, PIK3CG)↓ | | |
| 12 | MYB↑, ARAF↑ | TP53, | | (ELK1, ARAF, HMGA2, FGFR2, | Nab | PR |
| | | CDH5 | | GLI1, SHH, TGFB2, PDGFRB, | paclitaxel + | nodes |
| | | | | PDGFRA)↑ | capecitabine | |
| | | | | CDKN2A↓ | | |
| 13 | CADM2↑, FOXI1↑, | TP53, | | DLL1↑ CCNB3↑ FGFR2↑ | Iniparib + | PR |
| | TNNI2↓ | STAM2 | | SMAD6↑ | gemcitabine + | liver |
| | | HMMR | | SFRP2↓ PTCH2↓ | carboplatin | |
| 14 | FOXM1↑, PTPRM↓ | TP53, | | IGFBP3↑ PRKCG↑ NRG4↑ | BEZ 235 | SD |
| | SMARCA↓4RUNX1↓ | STAT1 | | RG1↑ | | 21% |
| | | | | CDKN2A↓ | | reduction |
| | | | | | | in nodes |

Table 21. Key Genomic alterations, therapeutic interventions, and outcomes.
* From outlier analysis with log fold change ≦ or ≧ 2.0, and p-value p ≦ 0.05
\hat{ } Germline and somatic mutation detected
¹ Focal Amplificiation,
² Focal homozygous Deletion;
CR: Complete Response;
PR: Partial Response;
SD: Stable Disease;
PD: Progressive Disease.

### Detailed Description of Several Patients

mTNBC2

This was a 59 year old Caucasion woman diagnosed in 2009 with a 16 cm left breast grade 3 triple negative invasive ductal cancer with skin erosion and left axillary adenopathy. Staging studies showed hilar, lung and liver metastases. Family history was unknown as she was adopted; BRCA ½ testing was not done. She had a partial response with gemcitabine, carboplatin and iniparib on a phase III trial. Her disease remained unresectable, however, and she developed progressive disease in her breast after 7 months on this therapy. She was then treated with docetaxel/capecitabine without response. Tissue was then harvested from her breast mass for whole genome sequencing. During this time she was treated with paclitaxel plus bevacizumab and had a transient 2-month response. Sequencing revealed a high level BRAF amplification in this tumor, which was validated as being part of complex extrachromosomal double minute by FISH, likely leading to increased signaling of the RAS/RAF/MEK/ERK pathway, which would suggest the use of a RAF or MEK inhibitor. Furthermore, RNA-seq based expression analysis showed overexpression of BRAF and significant underexpression of INPP4B. Thus, a therapeutic strategy targeting both signaling pathways was supported.

In March, 2011, the patient began treatment on a Phase I study of the oral MEK inhibitor GSK1120212 in combination with the oral AKT inhibitor GSK 2141795, based on the findings of a high level BRAF amplicon and decreased levels of INPP4B in her cancer on whole genome sequencing that were validated in a CLIA laboratory. By April, 2011, her breast mass had nearly completely regressed leaving an open wound on her chest wall. She developed toxicities including skin ulceration, diarrhea, anorexia and significant fatigue. After 3 months on therapy the patient had a seizure and was found to have a hemorrhagic brain metastasis and she discontinued the investigational therapies.

mTNBC6

TNBC-006 is a 32 year old African American woman who presented in June 2009 with a locally advanced, high grade, right side TNBC, and received preoperative TAC (docetaxel, doxorubicin, cyclophosphamide) chemotherapy. At bilateral mastectomy she had 7 cm residual disease in her right breast with extensive lymphphovascular invasion and 2 positive axillary lymph nodes with extranodal extension. She was treated with chest wall and regional radiation therapy along with weekly carboplatin during the radiation. Two paternal aunts and her maternal grandmother and aunt had postmenopausal breast cancer. BRCA½ testing was negative for a germline mutation. In September, 2010 her disease recurred diffusely over chest wall including a 4×4 cm protruberant mass on her left chest wall that was excised for whole genome sequencing that was biopsy confirmed as highly proliferative triple negative carcinoma. PET CT scan showed a right pleural effusion, small lung metastases and left hilar adenopathy. She was randomized to receive paclitaxel, bevacizumab and everolimus on a phase II study, had a complete response of 12 months duration.

A focal (212 kb) homozygous deletion at 4q31.2 encompassing the FBXW7 locus was detected. Furthermore, both differential expression methods showed significant under expression of FBXW7 in mTNBC6. FBXW7 is a tumor suppressor that targets mTOR for degradation through direct interaction, and cell lines harboring FBXW7 mutations and deletions were highly sensitive to the mTOR inhibitor rapamycin. A second hit in the PI3K/AKT/mTOR pathway was also detected in this tumor in the form of two independent inversion events encompassing exons, and a nonsynonymous SNV within the INPP5A gene. INPP5A encodes inositol polyphosphate-5-phosphatase F, which decreases AKT and GSK3B phosphorylation through its 5-phosphatase activities on phosphatidylinositol 4,5-bisphosphate (PIP2) and phosphatidylinositol 3,4,5-triphosphate (PIP3). This patient has since progressed to her liver. She has had stable disease on treatment with gemcitabine and carboplatin. The initial significant response to combination paclitaxel, bevacizumab and everolimus was believed to be associated at least in part with targeting of mTOR by everolimus, where mTOR activity was driven by loss of FBXW7 and possibly additional PI3K/AKT/mTOR signaling associated with INPP5A mutation.

mTNBC9

TNBC-009 was a 38 year old Caucasion woman who presented in May, 2008 with a T4 right breast mass and palpable right axillary adenopathy. Biopsy revealed high grade TNBC with a ki-67 of 78% and PET CT scan showed diffuse adenopathy in right axilla, bilateral supraclavicular fossa and hila, as well as mediastinum. She had a complete response on exam and PET CT with preoperative AC/T (doxorubicin, cyclophosphamide then paclitaxel) chemotherapy, and at bilateral mastectomy had minimal residual disease in her breast and axilla. She was treated with wide-field radiotherapy and 4 cycles of adjuvant cisplatin. Her maternal lineage included her grandfather with gastric cancer, aunt with colon cancer and uncle with melanoma. BRCA½ testing did not show a germline mutation. In April, 2010 she underwent resection of a solitary left occipital brain metastasis and declined radiotherapy. Seven months later she developed left posterior cervical lymphadenopathy, which was partially excised for whole genome sequencing, as well as recurrent brain metastases and underwent whole brain radiotherapy. She had a complete response documented on PET CT scan with 4 months of treatment with gemcitabine, carboplatin, iniparib on an expanded access protocol. She stopped treatment for family reasons and two months later developed leptomeningeal metastases.

Sequencing analysis revealed a somatic event in multiple genes encoding proteins involved in DNA repair, double strand break repair, and homologous recombination repair. A class of small molecule inhibitors are in mature clinical development that target DNA repair enzymes including Poly-ADP polymerases PARP-1 and PARP-2 are believed to be active in advanced basal-like triple negative breast tumors with defects in DNA repair genes including those tumor arising in patients with inherited BRCA1 or BRCA2 mutations. It is possible that the effectiveness of the therapeutic combination that included Iniparib may have been due to the convergence of mutations in double strand break repair and homologous recombination repair.

PI3K/AKT/mTOR and RAS/RAF/MEK/ERK Vulnerabilities in mTNBC

Five independent mTNBC (Table 18) described in this report demonstrated several biologically and potentially clinically relevant somatic alterations in genes involved in PI3K/AKT/mTOR and/or RAS/RAF/MEK/ERK signaling pathways (FIG. 10). Two tumors contained single events including mTNBC10 (focal KRAS amplification) and mTNBC12 (focal ARAF amplification). However, in at least three of these tumors (mTNBC1, mTNBC2, mTNBC6), multiple events supporting concomitant activation of both signaling pathways were observed. Targeting either pathway alone may not be sufficient in these cases, where molecular concepts supporting dual activation of both RAS/RAF/MEK/ERK and PI3K/AKT/mTOR signaling might suggest the use of a combination regimen targeting both signaling nodes. Both tumors demonstrating dual pathway alterations in this study described herein have ontologies indicative of basal-like tumors.

This study demonstrated benefits of understanding the comprehensive genomic framework of a tumor through whole genome and transcriptome analysis. For example, the single observation of high-level BRAF amplification in mTNBC2 is therapeutically relevant, but as in the context of wildtype BRAF, it is important to note that RAF inhibitors can enhance tumor growth in a RAS dependent manner, in contrast to MEK inhibitors. Thus, as mTNBC2 had gross wildtype BRAF amplification, a PI3K/AKT inhibitor in combination with a MEK inhibitor might be more effective than a combination including a RAF inhibitor, especially in light of additional alterations in the PI3K/AKT/mTOR pathway that were detected in this tumor. This supports the contention that understanding full genomic context may be prudent as the field continues to explore the effectiveness of prospective selection of therapeutic strategies through genomic interrogation.

In summary, NGS technologies and analytical tools were applied to perform a comprehensive genomic survey to elucidate the lexicon of somatic events occurring in an ethnicity matched cohort of 14 mTNBC. This study is incredibly integrative, where base level whole genome and transcriptome analysis were combined, and extremely comprehensive molecular data with deep clinical clinical history and outcomes information were integrated. Through this study the presence of alterations occurring within the genomes and transcriptomes of these tumors were validated and confirmed including common TP53 mutation, copy number changes (i.e. CTNNA1 homozygous deletion), and enrichment of gene expression ontologies associated with cell cycle control and mitosis. Additional genes that may play a role in mTNBC were also discovered. There was equal representation of both African American and Europea American patients, and at least one event (CTNNA1 homozygous deletion) may be enriched among women of recent west African descent. Furthermore the study revealed genomic concepts that may have significant downstream therapeutic implications such as dual activation of RAS/RAF/MEK/ERK and PI3K/AKT/mTOR signaling pathways in mTNBC, suggesting that combination trials using inhibitors of both signaling nodes should be initiated specifically for patients diagnosed with mTNBC. These results add tremendously to our nascent understanding of how best to manage and treat these generally irremedial tumors.

2815626.1 70

What is claimed is:

1. A method for detecting existence of a triple negative breast cancer (TNBC) subtype in a tumor comprising analyzing the genome or transcriptome of a tumor tissue sample for the presence of a TNBC biomarker profile, wherein the presence of the TNBC biomarker profile indicates existence of the TNBC subtype in the tumor, wherein the TNBC biomarker profile comprises at least one alteration selected from the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene.

2. A method for determining sensitivity or resistance of tumor cells to a particular chemotherapy comprising assessing a tumor tissue sample for a triple negative breast cancer (TNBC) biomarker profile, wherein the presence of TNBC subtype in the tumor sample indicates sensitivity or resistance of the tumor to a particular chemotherapy, wherein the TNBC biomarker profile comprises at least one alteration selected from the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene.

3. A method for selecting a treatment for a subject with TNBC, comprising:
(a) obtaining a tumor tissue sample from the subject;
(b) obtaining the TNBC biomarker profile of the sample and grouping the subject to a TNBC subtype;
(c) determining sensitivity or resistance of the subject to a particular chemotherapy based on the relationship between the TNBC subtype and sensitivity or resistance to particular chemotherapies; and
(d) selecting a treatment based on the TNBC subtype.

4. The method of claim 2, wherein the TNBC subtype is characterized by a biomarker profile comprising BRAF amplification and INPP4B underexpression and is sensitive to treatment with both MEK and AKT inhibitors.

5. The method of claim 2, wherein the TNBC subtype is characterized by a biomarker profile comprising TOP2a and PBK overexpression and is sensitive to treatment with Eribulin.

6. The method of claim 2, wherein the TNBC subtype is characterized by a biomarker profile comprising IQGAP3 and AKT3 overexpression and INPP4B underexpression and is sensitive to treatment with BEZ235.

7. The method of claim 2, wherein the TNBC subtype is characterized by a biomarker profile comprising ALK overexpression and is sensitive to treatment with ALK inhibitor.

8. The method of claim 2, wherein the TNBC subtype is characterized by a biomarker profile comprising FBXW7 and INPP4B underexpression and is sensitive to treatment with Taxol, Avastin and Everolimus.

9. The method of claim 2, wherein the TNBC subtype is characterized by a biomarker profile comprising DNA repair related gene mutations and is sensitive to treatment with BSI 201, Gemcitabine and Carboplatin.

10. The method of claim 2, wherein the TNBC subtype is characterized by a biomarker profile comprising IQGAP3 overexpression, INPP5F inversion and NEDD4 mutation and is resistant to treatment with BEZ 235.

11. The method of claim 2, wherein the TNBC subtype is characterized by a biomarker profile comprising GART overexpression and is resistant to treatment with both MEK and AKT inhibitors.

12. The method of claim 3, further comprising administering an effective amount of both MEK and AKT inhibitors to the subject with a TNBC biomarker profile comprising BRAF amplification and INPP4B underexpression.

13. The method of claim 3, further comprising administering an effective amount of Eribulin to the subject with a TNBC biomarker profile comprising IQGAP3 and AKT3 overexpression and INPP4B underexpression.

14. The method of claim 3, further comprising administering an effective amount of ALK inhibitor to the subject with a TNBC biomarker profile comprising ALK overexpression.

15. The method of claim 3, further comprising administering effective amounts of Taxol, Avastin and Everolimus to the subject with a TNBC biomarker profile comprising FBXW7 and INPP4B underexpression.

16. The method of claim 3, further comprising administering an effective amount of combined BSI 201, Gemcitabine and Carboplatin to the subject with a TNBC biomarker profile comprising DNA repair related gene mutations.

17. The method of claims 3, wherein the TNBC biomarker profile comprises at least two alterations selected from the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion;
INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene.

18. The method of claims 3, wherein the TNBC biomarker profile comprises at least three alterations selected from the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion; INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene.

19. The method of claim 3, wherein the TNBC biomarker profile comprises at least four alterations of the group consisting of an RB1 gene deletion, a PTEN gene deletion, an ERBB4 gene deletion, an ABCB1 gene mutation, a SLC9A11 gene translocation, a NCO6A gene translocation, a chromosome 11 translocation, a chromosome 16 translocation, a NF1 gene deletion, a FBXW7 gene deletion;
INPP5F inversion; overexpression of AURKB, FOXM1, PLK1, AURKA genes, BRAF, ERAS, IQGAP3; and underexpression of PTEN, ERBB4, INPP4B, CTNNA1 and NOVA1 gene.

20. The method of claim 3, wherein the TNBC biomarker profile comprises a deletion of an exon in an RB1 gene.

21. The method of claim 3, wherein the TNBC biomarker profile comprises a deletion of exon 6 in a PTEN gene.

22. The method of claim 3, wherein the TNBC biomarker profile comprises a deletion in an ERBB4 gene.

23. The method of claim 3, wherein the TNBC biomarker profile comprises a mutation in an ABCB1 gene.

24. The method of claim 3, wherein the TNBC biomarker profile comprises a chromosome 11 translocation and/or a chromosome 16 translocation.

25. The method of claim 3, wherein the TNBC biomarker profile comprises a translocation of an SLC9A11 gene and a NCO6A gene.

26. The method of claim 3, wherein the TNBC biomarker profile comprises overexpression of a gene selected from the group consisting of AURKB, FOXM1, PLK1, AURKA, BRAF, ERAS, and IQGAP3.

27. The method of claim 3, wherein the TNBC biomarker profile comprises underexpression of a gene selected from the group consisting of PTEN, ERBB4, INPP4B and NOVA1.

28. The method of claim 3, wherein the TNBC biomarker profile comprises copy number variation correlated with altered gene expression.

29. The method of claims 3, wherein the TNBC biomarker profile comprises an overexpression of a FOXM1 gene.
